(19) United States
(12) Patent Application Publication
(10) Pub. No.: US 2025/0025513 A1
(43) Pub. Date: Jan. 23, 2025

(54) METHODS FOR TREATING NEUROLOGICAL DISEASES

(71) Applicant: Cytora Ltd., Yokneam (IL)

(72) Inventors: Sandu PITARU, Tel Aviv (IL); Haim GOLTSMAN, Gesher Haziv (IL); Belly KOREN, Shchenia (IL); Avital AMILOV, Kiriat Bialik (IL)

(21) Appl. No.: 18/909,327

(22) Filed: Oct. 8, 2024

Related U.S. Application Data
(63) Continuation-in-part of application No. 18/474,676, filed on Sep. 26, 2023, now Pat. No. 12,115,196.
(63) Continuation of application No. PCT/IL2024/050706, filed on Jul. 18, 2024.
Application No. PCT/IL2024/050706 is a continuation of application No. 18/474,676, filed on Sep. 26, 2023, now Pat. No. 12,115,196.
(60) Provisional application No. 63/514,580, filed on Jul. 20, 2023.

(51) Int. Cl. A61K 35/38; A61K 9/00; A61M 5/32

(52) U.S. Cl. CPC A61K 35/38; A61K 9/0019; A61M 5/32

(57) ABSTRACT

The present invention provides methods of treating neurological diseases and disorders by intrathecal administration of cells, such as human stem cells, while the method provides a reduced rate of side effects associated with intrathecal administration.

### FIELD OF THE INVENTION

The present invention is related to methods of administering human therapeutic cells for the treatment of neurodegenerative diseases or disorders thereof.

### BACKGROUND OF THE INVENTION

Intrathecal (IT) administration is a well-known medical procedure whereby medication is delivered to the central nervous system. These medications include drugs, biologicals and therapeutic cells that are capable of releasing neurotrophic factors that aim at treating neurological diseases or disorders. IT delivery of therapeutic cells for the treatment of neurodegenerative diseases including Multiple System Atrophy (MSA) has been associated with adverse events, e.g., back pain, referral lower limb pain and thickening of the nerve roots in the proximity of the IT site of injection as disclosed by Magnetic Resonance Imaging (MRI) abnormalities (Singer et al. Neurology, 2019). The intensity and duration of these adverse events increased with dosage augmentation thereby limiting the therapeutic dose. The MRI analysis suggested that following the IT administration a significant number of cells remained clumped in the lumbar area of the spinal cord and cauda equina, at a considerable distance from the brain, a fact that might reduce the efficacy of this treatment. Alternative procedures for delivering cells to the central nervous system are by intra-carotid delivery. However, this approach is associated with mini-ischemic strokes due to small artery blockage by cell clumps. (Lee et al., Clinical Pharmacology and Therapeutics, 2008; Lee et al., Annals of Neurology, 2012).

Thus, there is still a need in the art to develop a safe and effective method of IT administration procedure that is capable of administering high doses of cells with therapeutic efficacy for the treatment of neurodegenerative diseases while minimizing the side effects associated with the IT administration procedure.

### SUMMARY OF THE INVENTION

The present invention provides a method of treating a neurological disease or disorder by intrathecally administering a therapeutic cell suspension to a patient in need thereof while overcoming previously reported side effects associated with intrathecal administration. According to one aspect, the present invention provides a method for treating a neurological disease or disorder in a subject in need thereof comprising the steps of intrathecally (IT) administering from 5 to 40 ml of a therapeutic cell suspension at a rate of from 0.1 to 2 ml/min, wherein the amount of the administered cells is from 1×10⁶ to 1×10⁹ and/or the therapeutic cell suspension comprises from 0.1×10⁵ to 5×10⁷ cells/ml. According to some embodiments, the method comprises performing a lumbar puncture prior to IT administering the therapeutic cell suspension. Thus, according to one some embodiments, the present invention provides a method for treating a neurological disease or disorder in a subject in need thereof comprising the steps of:
(i) performing a lumbar puncture; and
(ii) intrathecally administering from 5 to 40 ml of a therapeutic cell suspension at a rate of from 0.1 to 2 ml/min,
   wherein the amount of the administered cells is from 1×10⁶ to 1×10⁹ and/or the therapeutic cell suspension comprises from 0.1×10⁵ to 5×10⁷ cells/ml. In some examples, the rate of administering is from 0.1 to 1.5 ml/min or from 0.5 to 1.5 ml/min. In some examples, the amount of the administered cells is from 1×10⁶ to 5×10⁸. In some examples, the method comprises intrathecally administering from 5 to 35 ml of the therapeutic cell suspension. In some examples, the method comprises intrathecally administering from 5 to 30 ml of the therapeutic cell suspension. In some examples, the method comprises intrathecally administering from 5 to 25 ml of the therapeutic cell suspension. In some examples, the therapeutic cell suspension comprises from 0.5×10⁵ to 5×10⁷ cells/ml. In some examples, the therapeutic cell suspension comprises from 0.5×10⁶ to 5×10⁷ cells/ml. In some examples, the method further comprises drawing a volume from 5 to 40 ml of cerebrospinal fluid (CSF) prior to IT administering the therapeutic cell suspension. In other examples, the method further comprises drawing a volume from 5 to 40 ml of cerebrospinal fluid (CSF) at step (i) wherein the volume of the administered therapeutic cell suspension is equal to or up to +5 ml of the volume of CSF drawn in step (i). In some examples, the method comprises drawing a volume from 5 to 35 ml of cerebrospinal fluid (CSF) prior to IT administering the therapeutic cell suspension, e.g. at step (i). In some examples, the method comprises drawing a volume from 5 to 30 ml of cerebrospinal fluid (CSF) prior to IT administering the therapeutic cell suspension, e.g, at step (i). In some examples, the method comprises drawing a volume from 5 to 25 ml of cerebrospinal fluid (CSF) prior to IT administering the therapeutic cell suspension, e.g. at step (i). Thus, in some examples, the present invention provides a method for treating a neurological disease or disorder in a subject in need thereof comprising the steps of:
(i) performing a lumbar puncture and drawing a volume from 5 to 25 ml of cerebrospinal fluid (CSF); and
(ii) intrathecally administering a therapeutic cell suspension at a rate of from 0.1 to 1.5 ml/min,
   wherein the volume of the administered therapeutic cell suspension is equal to or up to ±5 ml the volume of CSF drawn in step (i). According to some examples, the amount of the administered cells is from 1×10⁶ to 5×10⁸. According to some examples, the therapeutic cell suspension comprises from 1×10⁶ to 5×10⁷ cells/ml. According to some examples, the therapeutic cell suspension comprises from 1×10⁶ to 3×10⁷ cells/ml. According to some examples, the therapeutic cell suspension comprises from 1×10⁶ to 2×10⁷ cells/ml. According to some examples, the therapeutic cell suspension comprises from 1×10⁶ to 1×10⁷ cells/ml. According to some examples, the therapeutic cell suspension comprises from 1×10⁵ to 2×10⁷ cells/ml. In some examples drawing CSF and administering the therapeutic cell suspension is performed simultaneously.

According to one example, the neurological disease or disorder is a neurodegenerative disease. According to some examples, the neurodegenerative disease is selected from the group consisting of Multiple System Atrophy, Alzheimer's Disease, Pick Disease, Parkinsonism, Idiopathic Parkinson's Disease, Progressive Supranuclear Palsy, Corticobasal Degeneration, Striatonigral Degeneration, Shy-Drager Syndrome, Olivopontocerebellar Atrophy, Huntington Disease, Spinocerebellar Ataxias, Friedreich Ataxia, Ataxia-Telangiectasia, Amyotrophic Lateral Sclerosis, Bulbospinal Atrophy, Spinal Muscular Atrophy, and combinations thereof. According to a more specific example, the neurodegenerative disease is multiple system atrophy. According to some embodiments, the neurological disease or disorder is selected from the group comprising of a trauma to the central nervous system (CNS), brain trauma, trauma to the spinal cord, trauma to the spinal ganglions, and a chronic disease affecting the central nervous system. Non-limiting examples of such diseases are diabetes, lupus, post-atherosclerotic stroke and post-hemorrhagic stroke caused by intervertebral disc pathologies.

According to another aspect, the present invention provides a method for intrathecally administering a therapeutic cell suspension to a subject in need thereof comprising the steps of:
(i) performing a lumbar puncture using a spinal needle and drawing a volume of from 5 to 40 ml of cerebrospinal fluid (CSF); and
(ii) intrathecally administering a therapeutic cell suspension at a rate of from 0.1 to 1.5 ml/min
   wherein the volume of the administered therapeutic cell suspension is equal to or about ±5 ml the volume of CSF drawn in step (i). According to some examples, the amount of the administered cells is from 1×10⁶ to 1×10⁹. According to some examples, the therapeutic cell suspension comprises from 1×10⁵ to 5×10⁷ cells/ml. According to some examples, the therapeutic cell suspension comprises from 1×10⁶ to 5×10⁷ cells/ml.

According to any one of the above aspects and examples, the rate of administration of the therapeutic cell suspension in step (ii) is from 0.5 to 1.5 ml/min.

According to any one of the above aspects and examples, the method comprises administering from 1×10⁷ to 1×10⁸ cells.

According to any one of the above aspects and examples, the volume of the therapeutic cell suspension administered in step (ii) equals to the volume of CSF drawn in step (i).

According to any one of the above aspects and examples, the concentration of cells in the therapeutic cell suspension is from 0.5×10⁶ to 5×10⁷ cells/ml, 1×10⁶ to 5×10⁷ cells/ml, from 1×10⁶ to 4×10⁷ cells/ml, from 1×10⁶ to 2×10⁷ cells/ml or from 1×10⁶ to 10×10⁶ cells/ml.

According to any one of the above aspects and examples, the therapeutic cell suspension is administered via the spinal needle. According to any one of the above aspects and examples, the CSF is drawn via the spinal needle. According to a more specific example, the therapeutic cell suspension is mixed during administration to prevent the clumping of cells. According to some embodiments, cell clumping comprises cell clumping adjacent to the injection site. According to some embodiments, cell clumping comprises cell clumping adjacent to the nerve roots of the cauda equina near the injection site. According to yet another example, the mixing comprises rotating or tilting the device comprising the therapeutic cell suspension manually or by a designated automatic device.

According to any one of the above aspects and examples, the method is characterized by a lower rate of side effects associated with intrathecal administration of cells in comparison to a corresponding method comprising intrathecal administering cells at a rate higher than 2 ml/min. According to other examples, the method is characterized by a lower rate of side effects associated with intrathecal administration of cells in comparison to a corresponding method comprising intrathecal administering a therapeutic cell suspension comprising more than 2×10⁷ cells/ml at a rate higher than 2 ml/min. According to yet another example, the side effects are selected from the group comprising of back pain, pain in lower limbs, cell clumping, thickening or mild enhancement of cauda equina nerve roots near the injection site, and any combinations thereof. According to some embodiments, cell clumping comprises cell clumping adjacent to the nerves near the injection site. According to some embodiments, cell clumping comprises cell clumping adjacent to the nerve roots of the cauda equina near the injection site. According to a more specific example, the therapeutic cell suspension comprises a plurality of cells selected from human stem cells, naïve (undifferentiated) adult stem cells, progenitor cells, differentiated cells derived from embryonic or adult stem cells, induced pluripotent stem cells, and combinations thereof. According to yet another example, the cells are the human stem cells. According to a more specific example, the human stem cells are derived from lamina propria of the oral mucosa. According to a more specific example, the cells are derived from the lamina propria of the lining and masticatory oral mucosa. According to yet another example, the cells are characterized by simultaneously expressing the following markers: Oct-4, SSEA4, Nanog, Sox2, KLF4, c-MYC, nestin, β-III tubulin, p75, CD29, CD 73, CD90, CD105, and CD166. According to a more specific example, are characterized by simultaneously expressing the following markers: KLF4, c-MYC, nestin, β-III tubulin, and p75, and wherein the cells are negative for CD45 and CD31.

According to another aspect, the present invention provides a therapeutic cell suspension for use in a method of treatment of a neurological disease or disorder in a subject in need thereof wherein the method comprises intrathecally administering of from 5 to 40 ml of the therapeutic cell suspension at a rate of from 0.1 to 2 ml/min and wherein (a) the amount of the administered cells is from 1×10⁶ to 1×10⁹; (b) the concentration of cells in the therapeutic cell suspension is from 1×10⁵ to 2×10⁷ cells/ml; or (c) both (a) and (b). In some examples, the method comprises intrathecally administering from 5 to 35 ml of the therapeutic cell suspension at a rate of from 0.1 to 2 ml/min. In some examples, the method comprises intrathecally administering from 5 to 30 ml of the therapeutic cell suspension at a rate of from 0.1 to 2 ml/min. In some examples, the method comprises intrathecally administering of from 5 to 25 ml of the therapeutic cell suspension at a rate of from 0.1 to 2 ml/min. All examples and embodiments related to a method of treatment of a neurological disease or disorder are contemplated in the aspect.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention provides a method of intrathecally administering a therapeutic cell suspension into the CSF of a patient in need thereof suffering from a neurological disease or disorder. It was unexpectedly found that the slow administration rate and/or the relatively low concentration of cells optionally together with the Trendelenburg position allow to overcome previously reported side effects associated with intrathecal administration of cells such as back pain, pain in lower limbs or those shown as lumbar MRI abnormalities such as cell clumping adjacent to the nerve roots near the injection site e.g. nerve roots of the cauda equina near the injection site, thickening or mild enhancement of cauda equina nerve roots near the injection site suggesting accumulation of cells in the injected area. Cell clumping might be associated with adverse events of considerable intensity and duration in addition to reduced therapeutic efficacy. Other reported side effects associated with intrathecal cell administration at a relatively fast rate and high concentration, included post-operative back pain of considerable intensity and duration with referral pain in the lower limbs. This problem is common to IT administration of cells regardless of which type of cell is administered. Without being bound to any particular theory it is assumed that the method of administration facilitates the dispersion of the cells within the cerebrospinal fluid (CSF) and their movement in a cranial direction. The provided mode of administration thereby prevents cell clumping adjacent to the site of administration and allows dispersing cells cranially, and their attachment to the surface of the arachnoid mater and pia mater as close as possible to the brain and along the length of the spinal cord. Subsequently, a higher number of cells reach its target and consequently enhance the desired therapeutic effect. The present invention provides methods for treating neurological diseases treatable by intrathecal administration of a therapeutic cell suspension implementing slow administration of the cell suspension to a patient in need thereof suffering from a neurological disease or disorder.

The present invention provides a method of administration with the following advantages:
(i) Locating the therapeutic cells as close as possible to the affected tissues within the brain or spinal cord.
(ii) Higher spreading of the cells allows for higher secreting surface and thereby the secretion of higher amounts of trophic factors.
(iii) Preventing cell accumulation in the administration area which is at the far (caudal) end of the CNS.
(iv) The use of higher concentrations and consequently higher doses of therapeutic cells with minimal side effects.

According to one aspect, the present invention provides a method for treating a neurological disease or disorder in a subject in need thereof comprising intrathecally administering from 5 to 40 ml of a therapeutic cell suspension at a rate of from 0.1 to 2 ml/min, wherein the amount of the administered cells is from 1×10⁶ to 5×10⁸ and/or wherein the concentration of cells in the therapeutic cell suspension is from 0.5×10⁵ to 2×10⁷ cells/ml.

According to another aspect, the present invention provides a therapeutic cell suspension for use in treating a neurological disease or disorder in a subject in need thereof, wherein the use comprises intrathecally administering from 5 to 40 ml of a therapeutic cell suspension at a rate of from 0.1 to 1.5 ml/min, wherein the amount of the administered cells is from 1×10⁶ to 5×10⁸.

The below embodiments refer both to the method for treating and to a therapeutic cell suspension for use in treating a neurological disease or disorder.

In some embodiments, the method or the use comprises administering from 5 to 35 ml of the therapeutic cell suspension. In some embodiments, the method or the use comprises administering from 5 to 30 ml of the therapeutic cell suspension. In some embodiments, the method or the use comprises administering from 5 to 25 ml of the therapeutic cell suspension. In some embodiments, the method or the use comprises administering from 5 to 20 ml of the therapeutic cell suspension. In some embodiments, from 5 to 15 ml of the therapeutic cell suspension are administered. In some embodiments, from 10 to 25 ml of the therapeutic cell suspension are administered. In some embodiments, from 15 to 25 ml of the therapeutic cell suspension are administered. In some embodiments, from 10 to 20 ml of the therapeutic cell suspension are administered. In some embodiments, the concentration of cells in the therapeutic cell suspension is from 1×10⁵ to 2×10⁷ cells/ml. In some embodiments, the concentration of cells in the therapeutic cell suspension is from 0.5×10⁶ to 2×10⁷ cells/ml. In some embodiments, the concentration of cells in the therapeutic cell suspension is from 1×10⁶ to 2×10⁷ cells/ml. In some embodiments, the concentration of cells in the therapeutic cell suspension is from 1×10⁶ to 10×10⁶ cells/ml. According to some embodiments, the present invention provides a method for treating a neurological disease or disorder in a subject in need thereof comprising intrathecally administering from 5 to 40 ml of a therapeutic cell suspension at a rate of from 0.1 to 2 ml/min, wherein the amount of the administered cells is from 1×10⁶ to 5×10⁸. According to other embodiments, the present invention provides a method for treating a neurological disease or disorder in a subject in need thereof comprising intrathecally administering from 5 to 40 ml of a therapeutic cell suspension at a rate of from 0.1 to 2 ml/min, wherein the concentration of cells in the therapeutic cell suspension is from 1×10⁵ to 2×10⁷ cells/ml.

According to some embodiments, the method or the use comprises performing a spinal puncture prior to administration of the therapeutic cell suspension. According to some embodiments, the method comprises performing a lumbar puncture. As used herein, the term “lumbar puncture” refers to a spinal puncture between any two vertebrae of the spine. Therefore, according to one embodiment, the present invention provides a method for treating a neurological disease or disorder in a subject in need thereof comprising the steps of:
(i) performing a lumbar puncture; and
(ii) intrathecally administering from 5 to 40 ml of a therapeutic cell suspension at a rate of from 0.1 to 2 ml/min,

wherein the amount of the administered cells is from 1×10⁶ to 5×10⁸.

According to other embodiments, the present invention provides a method for treating a neurological disease or disorder in a subject in need thereof comprising the steps of:
(i) performing a lumbar puncture; and
(ii) intrathecally administering from 5 to 40 ml of a therapeutic cell suspension at a rate of from 0.1 to 2 ml/min,

wherein the concentration of cells in the therapeutic cell suspension is from 10⁵ to 2×10⁷ cells/ml.

According to some embodiments, the lumbar puncture is performed using a spinal needle. According to any one of the above embodiments, the method or the use further comprises drawing a volume from 5 to 40 ml or from 5 to 35 ml of cerebrospinal fluid (CSF) at step (i). According to any one of the above embodiments, the method or the use further comprises drawing a volume from 5 to 30 ml or from 5 to 25 ml of cerebrospinal fluid (CSF) at step (i). According to any one of the above embodiments, the method or the use further comprises drawing a volume from 10 to 40 ml or from 10 to 30 ml of cerebrospinal fluid (CSF) at step (i). According to any one of the above embodiments, the method or the use further comprises drawing a volume from 15 to 30 ml or from 10 to 20 ml of cerebrospinal fluid (CSF) at step (i). According to some embodiments, the volume of the administered therapeutic cell suspension is equal to or up to ±5 ml of the volume of CSF drawn in step (i). According to some embodiments, the administration and the drawing of the CSF are performed via the same puncture location. According to other embodiments, the administration and the drawing of the CSF are performed via the different puncture locations.

According to some embodiments, the present invention provides a method for treating a neurological disease or disorder in a subject in need thereof comprising the steps of:
(i) performing a lumbar puncture and drawing a volume of from 5 to 30 ml of cerebrospinal fluid (CSF); and
(ii) intrathecally administering a therapeutic cell suspension at a rate of from 0.1 to 2 ml/min,

wherein the volume of the administered therapeutic cell suspension is equal to or up to ±5 ml the volume of CSF drawn in step (i). According to one embodiment, the amount of the administered cells is from 1×10⁶ to 5×10⁸. Thus, according to some embodiments, the present invention provides a method for treating a neurological disease or disorder in a subject in need thereof comprising the steps of:
(i) performing a lumbar puncture and drawing a volume from 5 to 30 ml of cerebrospinal fluid (CSF); and
(ii) intrathecally administering a therapeutic cell suspension at a rate of from 0.1 to 2 ml/min,

wherein the volume of the administered therapeutic cell suspension is equal to or up to ±5 ml the volume of CSF drawn in step (i) and the amount of the administered cells is from 1×10⁶ to 5×10⁸. In some embodiments, the method or the use comprises drawing from 5 to 40 ml of CSF and/or intrathecally administering from 5 to 40 ml of the therapeutic cell suspension. In some embodiments, the method or the use comprises drawing from 5 to 25 of CSF and/or intrathecally administering from 5 to 25 of the therapeutic cell suspension. In some embodiments, the method or the use comprises drawing 10 to 25 of CSF and/or intrathecally administering from 10 to 25 of the therapeutic cell suspension. In some embodiments, the method or the use comprises drawing from 10 to 20 of CSF and/or intrathecally administering from 10 to 20 of the therapeutic cell suspension. According to some embodiments, the present invention provides a method for treating a neurological disease or disorder in a subject in need thereof comprising the steps of:
(i) performing a lumbar puncture and drawing a volume from 5 to 30 ml of cerebrospinal fluid (CSF); and
(ii) intrathecally administering a therapeutic cell suspension at a rate of from 0.1 to 2 ml/min,
   wherein the volume of the administered therapeutic cell suspension is equal to or up to ±5 ml the volume of CSF drawn in step (i) and the concentration of cells in the therapeutic cell suspension is from 1×10⁶ to 2×10⁷ cells/ml.

According to some embodiments, the neurological disease or disorder is a neurodegenerative disease or disorder. The terms “neurological disease or disorder” and “neural disease or disorder” may be used interchangeably. Typically, the term “neural” refers to nerves or groups of nerves. According to some embodiments, neurodegenerative disease or disorder is selected from the group consisting of a disease of basal ganglia and brain stem, a disease affecting the cerebral cortex, a spinocerebellar degeneration, a degenerative disease affecting motor neurons, or combinations thereof.

In some embodiments, the neurodegenerative disease is a disease of the basal ganglia and brain stem. According to some embodiments, the disease is Multiple System Atrophy. According to another embodiment, the disease is Parkinsonism. According to another embodiment, the disease is Idiopathic Parkinson's Disease. According to another embodiment, the disease is Progressive Supranuclear Palsy. According to another embodiment, the disease is Corticobasal Degeneration. According to another embodiment, the disease is Striatonigral Degeneration. According to another embodiment, the disease is Shy-Drager Syndrome. According to another embodiment, the disease is Olivopontocerebellar Atrophy. According to another embodiment, the disease is ALS. According to another embodiment, the disease is Huntington's Disease. According to some embodiments, the neurodegenerative disease is Multiple System Atrophy. According to some embodiments, the neurological disease or disorder is selected from the group consisting of trauma to the central nervous system (CNS), brain trauma, trauma to the spinal cord, trauma to the spinal ganglions, and a chronic disease affecting the central nervous system. According to some embodiments, the chronic disease affecting the central nervous system is selected from diabetes, lupus, post-atherosclerotic stroke, and post-hemorrhagic stroke caused by intervertebral disc pathologies. According to some embodiments, the present invention provides a method for treating Multiple System Atrophy in a subject in need thereof comprising intrathecally administering from 5 to 25 ml of a therapeutic cell suspension at a rate of from 0.1 to 2 ml/min, wherein the amount of the administered cells is from 1×10⁶ to 5×10⁸ and/or wherein the concentration of cells in the therapeutic cell suspension is from 0.5×10⁶ to 2×10⁷ cells/ml.

In some embodiments, the neurodegenerative disease is a disease affecting the cerebral cortex. According to some embodiments, the disease is Alzheimer's Disease. According to another embodiment, the disease is Pick Disease. In some embodiments, the neurodegenerative disorder is a Spinocerebellar degeneration. According to some embodiments, the disorder is Spinocerebellar Ataxias. According to another embodiment, the disorder is Friedreich Ataxia. According to another embodiment, the disorder is Ataxia-Telangiectasia.

In some embodiments, the neurodegenerative disease is a degenerative disease affecting motor neurons. According to some embodiments, the disease is Amyotrophic Lateral Sclerosis. According to another embodiment, the disease is Bulbospinal Atrophy. According to some embodiments, the disease is Spinal Muscular Atrophy.

The term “treating” a condition or patient refers to taking steps to obtain beneficial or desired results, including clinical results. Beneficial or desired clinical results include, but are not limited to, ameliorating, abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting the development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s). The term “treatment” as used herein refers to both therapeutic treatment and prophylactic or preventative measures. Those in need of treatment include those already with the disorder as well as those in which the disorder is to be prevented.

The composition of the present invention may be administered by any known method into the central neural system. The term “administering” or “administration of” a substance, a compound or an agent to a subject can be carried out using one of a variety of methods known to those skilled in the art. According to some embodiments, the administration is intrathecal administration. The term “intrathecal administration” or “intrathecal injection” refers to an injection into the spinal canal (intrathecal space surrounding the spinal cord). Various techniques may be used including, without limitation, lumbar puncture or lateral cerebroventricular injection through a borehole or cisternal or the like. In some embodiments, “intrathecal administration” or “intrathecal delivery” according to the present invention refers to IT administration or delivery via the lumbar area or region, i.e., lumbar IT administration or delivery. In some embodiments, the term intrathecal administration refers to administration into the subarachnoid space.

A “therapeutically effective amount” of a drug or agent, e.g. therapeutic cell suspension is an amount of a drug or an agent that, when administered to a subject will have the intended therapeutic effect. The full therapeutic effect does not necessarily occur by the administration of one dose and may occur only after the administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentation.

In some examples, the present invention provides a method for intrathecally administering a therapeutic cell suspension to a subject in need thereof comprising the steps of:
(i) performing a lumbar puncture and drawing a volume of from 5 to 40 ml of cerebrospinal fluid (CSF); and
(ii) intrathecally administering a therapeutic cell suspension at a rate of from 0.1 to 2 ml/min
   wherein the volume of the administered therapeutic cell suspension is equal to or up to ±5 ml of the volume of CSF drawn in step (i), and wherein the amount of the administered cells is from 1×10⁶ to 5×10⁸.

According to any one of the aspects and embodiments of the application, step (i) comprises drawing from 5 to 35 ml of CSF. According to some embodiments, step (i) comprises drawing from 5 to 30 ml of CSF. According to some embodiments, step (i) comprises drawing from 5 to 25 ml of CSF. According to some embodiments, step (i) comprises drawing from 5 to 20 ml of CSF. According to some embodiments, step (i) comprises drawing from 10 to 20 ml of CSF. According to some embodiments, step (i) comprises drawing from 10 to 25 ml of CSF. According to some embodiments, step (i) comprises drawing from 10 to 30 ml of CSF. According to some embodiments, step (i) comprises drawing from 8 to 12 ml of CSF. According to some embodiments, step (i) comprises drawing from 12.5 to 17.5 ml of CSF. According to some embodiments, step (i) comprises drawing about 15 ml of CSF.

According to any one of the above embodiments, the volume of the therapeutic cell suspension administered in step (ii) equals to the volume of CSF drawn in step (i). According to any one of the above embodiments, the volume of the therapeutic cell suspension administered in step (ii) is ±1 to 5 ml of the volume of CSF drawn in step (i). According to any one of the above embodiments, the volume of the therapeutic cell suspension administered in step (ii) is ±1 ml, or ±2 ml, or ±3 ml, or ±4 ml, or ±5 ml of the volume of CSF drawn in step (i).

According to any one of the aspects and embodiments of the application, the volume of the therapeutic cell suspension administered in step (ii) is from 5 to 40 ml. According to any one of the above aspects and embodiments, the volume of the therapeutic cell suspension administered in step (ii) is from 5 to 35 ml. According to any one of the above aspects and embodiments, the volume of the therapeutic cell suspension administered in step (ii) is from 5 to 30 ml. According to any one of the above aspects and embodiments, the volume of the therapeutic cell suspension administered in step (ii) is from 5 to 25 ml. According to any one of the above aspects and embodiments, the volume of the therapeutic cell suspension administered in step (ii) is from 5 to 20 ml. According to any one of the above aspects and embodiments, the volume of the therapeutic cell suspension administered in step (ii) is from 10 to 20 ml. According to any one of the above aspects and embodiments, the volume of the therapeutic cell suspension administered in step (ii) is from 12.5 to 17.5 ml. According to any one of the above aspects and embodiments, the volume of the therapeutic cell suspension administered in step (ii) is about 15 ml. According to any one of the above aspects and embodiments, the volume of the therapeutic cell suspension administered in step (ii) is about 20 ml.

According to any one of the aspects and embodiments of the application, the rate of administration of the therapeutic cell suspension is from 0.1 to 1.5 ml/min. According to any one of the aspects and embodiments of the application, the rate of administration of the therapeutic cell suspension is from 0.5 to 1.5 ml/min. According to some embodiments, the rate of administration of the therapeutic cell suspension is from 0.6 to 1.3 ml/min. According to some embodiments, the rate of administration of the therapeutic cell suspension is from 0.8 to 1.2 ml/min. According to some embodiments, the rate of administration of the therapeutic cell suspension is about 1 ml/min. According to some embodiments, the administration of therapeutic cell suspension is performed at step (ii). According to some embodiments, the rate of administration of the therapeutic cell suspension at step (ii) is from 0.1 to 1.5 ml/min, from 0.5 to 1.5 ml/min, from 0.6 to 1.3 ml/min, from 0.8 to 1.2 ml/min or about 1 ml/min.

As used herein the term “therapeutic cell suspension” refers to a suspension of cells in a pharmaceutically acceptable carrier, wherein the cells have therapeutic properties and the suspension is intended for therapeutic purposes, typically administered through injection or infusion into a patient's body. This suspension contains a specific population of cells that possess therapeutic properties, such as stem cells, or other specialized cell types. These cells are isolated, processed and suspended in a suitable solution or medium to maintain their viability and functionality.

According to any one of the aspects and embodiments of the invention, the therapeutic cell suspension comprises cells selected from the group consisting of naïve (undifferentiated) adult stem cells, progenitor cells, differentiated cells derived from embryonic or adult stem cells, or the progenitors of embryonic stem cells or adult stem cells, induced pluripotent stem cells and their progenies and derivatives, genetically manipulated cells transduced to express specific trophic factors or therapeutic factors, tissue-specific differentiated cells obtained by genetic engineering, and combinations thereof. According to some embodiments the therapeutic cell suspension comprises of naïve (undifferentiated) adult stem cells. According to other embodiments, induced pluripotent stem cells encompass also the derivatives of the induced pluripotent stem cells. The term “derivatives of the induced pluripotent stem cells” refers also to all compounds that the cells secrete, i.e. secretome derived thereof. According to other embodiments, the therapeutic cell suspension comprises progenitor cells. According to other embodiments, the therapeutic cell suspension comprises neural progenitor cells. According to other embodiments the therapeutic cell suspension comprises cells engineered to express neurotrophic factors. According to other embodiments the therapeutic cell suspension comprises differentiated cells derived from embryonic or adult stem cells. According to other embodiments the therapeutic cell suspension comprises induced pluripotent stem cells. According to other embodiments, the therapeutic cell suspension comprises stem cells selected from hematopoietic stem cells, mesenchymal stem cells, neural stem cells, and epithelial stem cells. According to other embodiments the therapeutic cell suspension comprises mesenchymal stromal cells, e.g. bone marrow mesenchymal stromal cells. According to other embodiments, the therapeutic cell suspension comprises neural crest-derived stem cells and their progenies derived from embryonic or adult tissues. According to other embodiments the therapeutic cell suspension comprises genetically engineered cells.

In some embodiments, the cells are human stem cells. According to some embodiments, the therapeutic cell suspension comprises human oral mucosa stem cells. According to some embodiments the therapeutic cell suspension comprises human stem cells derived from the lamina propria of the oral mucosa (hOMSC). According to some embodiments, the cells are derived from the lamina propria of the lining and masticatory oral mucosa. In some embodiments, the cells are derived from the oral mucosa of the gingiva. According to some embodiments, the cells are characterized by simultaneously expressing the following markers: OCT-4, SSEA4, NANOG, SOX2, KLF4, c-MYC, nestin, β-III tubulin, p75, CD29, CD 73, CD90, CD105, and CD166. According to some embodiments, the cells are characterized by simultaneously expressing the following markers KLF4, c-MYC, nestin, β-III tubulin, and p75 and being negative for CD45 and CD31. According to some embodiments, the cells are characterized by simultaneously expressing the following markers: OCT-4, SSEA4, NANOG, SOX2, KLF4, c-MYC, nestin, β-III tubulin, p75 CD29, CD 73, CD90, CD105, and CD166; the cells are negative for CD45 and CD31. According to some embodiments, the cells are isolated cells. According to some embodiments, the cells are pluripotent or multipotent stem cells.

The term “oral mucosa” refers to the mucosal lining of the oral cavity, namely: the checks and the alveolar ridge including the gingiva and the palate, the tongue, the floor of the mouth and the oral part of the lips. Oral mucosa is the mucosal lining the oral cavity, namely: the checks and the alveolar ridge including the gingiva and the palate, the tongue, the floor of the mouth and the oral part of the lips. Oral mucosa consists of an epithelial tissue of ectodermal origin and the lamina propria (LP) which is a connective tissue of ectomesenchymal origin. Similarly, to the ectomesenchymal origin of connective tissues in the oral cavity, cells of the oral mucosa lamina propria (OMLP) originate from the embryonic ectodermal neural crest.

The term “hOMSC” refers to human stem cells derived from the lamina propria of the oral mucosa. According to some embodiments, the hOMSC are characterized by simultaneously expressing the following markers KLF4, c-MYC, β-III tubulin, p75 and nestin, and being negative for CD45 and CD31. According to some embodiments, hOMSC are characterized by simultaneously expressing the following markers: OCT-4, SSEA4, NANOG, SOX2, KLF4, c-MYC, nestin, β-III tubulin, p75, CD29, CD 73, CD90, CD105, and CD166; the cells being negative for CD45 and CD31.

The term “Stem cells” (SC) refers to undifferentiated cells, which can give rise to a succession of mature functional cells. Differentiated stem cells or stem cell population according to the present invention refer to partially or fully differentiated stem cells or naïve stem cell population.

The term “Embryonic stem (ES) cells” refers to cells derived from the inner cell mass of the embryonic blastocysts that are pluripotent, thus possessing the capability of developing into any organ or tissue type or, at least potentially, into a complete embryo.

The term “Adult stem cells” refers to post-natal stem cells derived from tissues, organs or blood of an organism after its birth.

The term “Pluripotent stem cells” refers to stem cells capable of generating the three embryonic cell layers and their derivatives cell lineages and tissues;

The term “Multipotent stem cells” refers to stem cells capable of forming multiple cell lineages that constitute an entire tissue or organ.

According to some embodiments, the use or method comprises administering from 1×10⁷ to 5×10⁸ of cells. According to some embodiments, use or the method comprises administering from 15×10⁶ to 500×10⁶ of cells. According to some embodiments, the use or method comprises administering from 20×10⁶ to 150×10⁶ of cells. According to some embodiments, the use or method comprises administering from 30×10⁶ to 100×10⁶ of cells. According to some embodiments, the use or method comprises administering from 30×10⁶ to 80×10⁶ of cells. According to some embodiments, the use or method comprises administering about 37.5×10⁶ cells. According to some embodiments, the use or method comprises administering about 75×10⁶ cells. According to some embodiments, the cells are hOMSC.

In some embodiments, the therapeutic cell suspension comprises from 0.1×10⁵ to 5×10⁸ cells/ml. In some embodiments, the therapeutic cell suspension comprises from 0.5×10⁵ to 5×10⁸ cells/ml. In some embodiments, the therapeutic cell suspension comprises from 1×10⁵ to 5×10⁸ cells/ml. In some embodiments, the therapeutic cell suspension comprises from 0.1×10⁶ to 5×10⁸ cells/ml. In some embodiments, the therapeutic cell suspension comprises from 0.5×10⁶ to 5×10⁸ cells/ml. In some embodiments, the therapeutic cell suspension comprises from 1×10⁵ to 1×10⁷ cells/ml. In some embodiments, the therapeutic cell suspension comprises from 5×10⁵ to 5×10⁶ cells/ml. In some embodiments, the therapeutic cell suspension comprises from 1×10⁶ to 1×10⁷ cells/ml. In some embodiments, the therapeutic cell suspension comprises from 0.1×10⁶ to 2×10⁷ cells/ml. In some embodiments, the therapeutic cell suspension comprises from 0.5×10⁶ to 2×10⁷ cells/ml. In some embodiments, the therapeutic cell suspension comprises from 1×10⁶ to 2×10⁷ cells/ml. In some embodiments, the therapeutic cell suspension comprises from 1×10⁶ to 10×10⁶ cells/ml. In some embodiments, the therapeutic cell suspension comprises from 1×10⁶ to 7×10⁶ cells/ml. In some embodiments, the therapeutic cell suspension comprises from 3×10⁶ to 7×10⁶ cells/ml. In some embodiments, the therapeutic cell suspension comprises from 1×10⁶ to 6×10⁶ cells/ml. In some embodiments, the therapeutic cell suspension comprises from 1×10⁶ to 5×10⁶ cells/ml. In some embodiments, the therapeutic cell suspension comprises from 5×10⁵ to 5×10⁶ cells/ml. In some embodiments, the therapeutic cell suspension comprises from 2×10⁶ to 5×10⁶ cells/ml. In some embodiments, the therapeutic cell suspension comprises from 2.5×10⁶ to 4×10⁶ cells/ml. According to some embodiments, the cells are hOMSC.

According to some embodiments, the present invention provides a method for treating a neurological disease or disorder in a subject in need thereof comprising the steps of:
(i) performing a lumbar puncture and drawing a volume from 5 to 40 ml of cerebrospinal fluid (CSF); and
(ii) intrathecally administering a therapeutic cell suspension at a rate of from 0.1 to 1.5 ml/min,

wherein the volume of the administered therapeutic cell suspension is equal to or up to ±5 ml the volume of CSF drawn in step, and wherein (a) the therapeutic cell suspension comprises from 1×10⁶ to 5×10⁸ cells, (b) the concentration of cells in the therapeutic cell suspension is from 1×10⁵ to 2×10⁷ cells/ml, or (c) both (a) and (b). According to some embodiments, the use or method comprises drawing a volume from 5 to 35 ml or from 5 to 30 ml of cerebrospinal fluid (CSF). According to some embodiments, the use or method comprises drawing a volume from 5 to 25 ml of cerebrospinal fluid (CSF). According to some embodiments, the method or the use comprises drawing a volume from 10 to 25 ml of cerebrospinal fluid (CSF). According to some embodiments, the use or method comprises drawing a volume from 10 to 20 ml of cerebrospinal fluid (CSF). According to some embodiments, the cells are hOMSC. According to one embodiment, the method or the use comprises administering from 1×10⁶ to 5×10⁸ cells and the therapeutic cell suspension comprises from 0.5×10⁶ to 2×10⁷ cells/ml. According to one embodiment, the method or the use comprises administering the therapeutic cell suspension comprising from 1×10⁶ to 2×10⁷ cells/ml.

According to some embodiments, the present invention provides a method for treating a neurodegenerative disease or disorder in a subject in need thereof comprising intrathecally administering from 5 to 25 ml of a therapeutic cell suspension comprising hOMSC at a rate of from 0.1 to 2 ml/min, wherein the amount of the administered cells is from 10×10⁶ to 200×10⁶ and/or the therapeutic cell suspension comprises from 1×10⁶ to 2×10⁷ cells/ml. According to some embodiments, the method or the use comprises performing a lumbar puncture and drawing a volume from 5 to 25 ml of cerebrospinal fluid (CSF), wherein the volume of the administered therapeutic cell suspension is equal to or up to ±5 ml the volume of CSF drawn. According to some embodiments, the present invention provides a method for treating a neurodegenerative disease or disorder in a subject in need thereof comprising the steps of:
(i) performing a lumbar puncture and drawing a volume from 5 to 25 ml of cerebrospinal fluid (CSF); and
(ii) intrathecally administering a therapeutic cell suspension comprising hOMSC at a rate of from 0.1 to 2 ml/min,
   wherein the volume of the administered therapeutic cell suspension is equal to or up to ±5 ml the volume of CSF drawn in step (i), and wherein the amount of the administered cells is from 10×10⁶ to 200×10⁶ and/or the therapeutic cell suspension comprises from 1×10⁶ to 2×10⁷ cells/ml. In some embodiments, the method or the use comprises drawing from 10 to 20 ml of CSF. In one embodiment, the method comprises drawing about 15 ml of CSF. In one embodiment, the number of administered hOMSC is from 10×10⁶/ml to 100×10⁶/ml. In one embodiment, the concentration of hOMSC in the therapeutic cell suspension is from 1×10⁶ to 15×10⁶ cells. In one embodiment, the concentration of hOMSC in the therapeutic cell suspension is from 1×10⁶ to 10×10⁶ cells/ml. In one embodiment, the concentration of hOMSC in the therapeutic cell suspension is from 1×10⁶/ml to 10×10⁶/ml cells. According to some embodiments, the lumbar puncture is performed using a spinal needle.

According to some embodiments, the present invention provides a method for treating a neurodegenerative disease or disorder in a subject in need thereof comprising the steps of:
(i) performing a lumbar puncture using a spinal needle and drawing a volume of 15±5 ml of cerebrospinal fluid (CSF); and
(ii) intrathecally administering 15±5 ml of a therapeutic cell suspension comprising from 20×10⁶ to 100×10⁶ hOMSC at a rate of from 0.1 to 1.2 ml/min. According to some embodiments, the neurodegenerative disease is Multiple System Atrophy.

According to some embodiments, the present invention provides a method for treating a neurodegenerative disease or disorder in a subject in need thereof comprising the steps of:
(i) performing a lumbar puncture using a spinal needle and drawing a volume of 15±5 ml of cerebrospinal fluid (CSF); and
(ii) intrathecally administering 15±5 ml of a therapeutic cell suspension comprising from 1×10⁶ to 10×10⁶ hOMSC/ml at a rate of from 0.1 to 1.2 ml/min. According to some embodiments, the neurodegenerative disease is Multiple System Atrophy.

The described dosage refers to a dosage for an adult subject. The dosage of subjects belonging to other groups of subjects (e.g., children, toddlers, infants, newborns) may be adapted and calculated according to the ratio between the common range of the CSF in adults and the corresponding common range of the CSF in subjects of the group.

According to some embodiments, the therapeutic cell suspension comprises cells of an autologous nature. According to other embodiments, the therapeutic cell suspension comprises cells of allogeneic nature.

According to some embodiments, the therapeutic cell suspension is administered via the spinal needle.

According to some embodiments, the therapeutic cell suspension is mixed during administration to prevent the clumping of cells. According to some embodiments, the mixing comprises rotating or tilting the device (e.g. a syringe) comprising the therapeutic cell suspension. According to one embodiment, the mixing is performed manually. In other embodiments, the mixing is performed by a designated automatic device.

According to any one of the above embodiments, the method or the use further comprises placing the subject in Trendelenburg position.

According to any one of the aspects and embodiments of the application, the administration may be performed manually or by an automatic syringe pump/injector.

According to any one of the aspects and embodiments, the method or the use according to the present invention is characterized by a lower rate of side effects associated with intrathecal administration of cells. According to some embodiments, the method according to the present invention is characterized by a lower rate of side effects associated with intrathecal administration of cells in comparison to a corresponding method comprising administering cells at a rate higher than 1.5 or higher than 2 ml/min. According to some embodiments, the method according to the present invention is characterized by a lower rate of side effects associated with intrathecal administration of cells in comparison to a corresponding method comprising intrathecal administering a therapeutic cell suspension comprising more than 2×10⁷ cells/ml at a rate higher than 2 ml/min. The term “corresponding” refers to a method having the same steps and parameters except for the state one.

According to some embodiments, the side effects associated with intrathecal administration of cells are selected from the group comprising of back pain, pain in lower limbs, cell clumping, thickening or mild enhancement of the cauda equina nerve roots near the injection site, and any combinations thereof. According to some embodiments, cell clumping comprises cell clumping adjacent to the nerve roots of the cauda equina near the injection site. Therefore, according to some embodiments, the method of the present invention causes less of at least one of the following side effects: back pain, pain in lower limbs, cell clumping, thickening or mild enhancement of the cauda equina nerve roots near the injection site. According to one embodiment, the method described in this invention reduces the rate of occurrence of back pain. According to another embodiment, the method described in this invention reduces the rate of occurrence of pain in lower limbs. According to another embodiment, the method described in this invention reduces the rate of occurrence of cell clumping adjacent to the nerve roots of the cauda equina near the injection site. According to another embodiment, the method described in this invention reduces the rate of occurrence of thickening of the cauda equina nerve roots near the injection site. According to another embodiment, the method described in this invention reduces the rate of occurrence of enhancement of the cauda equina nerve roots near the injection site as detected e.g., by MRI.

According to some aspects, the present invention provides a therapeutic cell suspension for use in treating a neurological disease or disorder in a subject in need thereof, wherein the use comprises intrathecally administering from 5 to 40 ml of a therapeutic cell suspension at a rate of from 0.1 to 1.5 ml/min, wherein the amount of the administered cells is from 1×10⁶ to 5×10⁸ and/or the concentration of cells in the therapeutic cell suspension is from 1×10⁵ to 2×10⁷ cells/ml. All terms, embodiments and definitions disclosed in any one of the above aspects apply and are encompassed herein as well. According to some embodiments, the present invention provides a therapeutic cell suspension for use in a method of treating a neurological disease or disorder in a subject in need thereof, wherein the use intrathecally administering from 5 to 40 ml of a therapeutic cell suspension at a rate of from 0.1 to 1.5 ml/min, wherein the amount of the administered cells is from 1×10⁶ to 5×10⁸. According to some embodiments, the use comprises performing a lumbar puncture before intrathecally administering the therapeutic cell suspension. Thus, according to some embodiments, the present invention provides a therapeutic cell suspension for use in treating a neurological disease or disorder in a subject in need thereof, wherein the use comprising the steps of:
(i) performing a lumbar puncture; and
(ii) intrathecally administering from 5 to 40 ml of a therapeutic cell suspension at a rate of from 0.1 to 1.5 ml/min,

wherein the amount of the administered cells is from 1×10⁶ to 5×10⁸.

According to some embodiments, the present invention provides a therapeutic cell suspension for use in a method of treatment of a neurological disease or disorder in a subject in need thereof wherein the method comprises intrathecally administering of from 5 to 40 ml of the therapeutic cell suspension at a rate of from 0.1 to 2 ml/min and wherein (a) the amount of the administered cells is from 1×10⁶ to 5×10⁸; (b) the concentration of cells in the therapeutic cell suspension is from 1×10⁵ to 2×10⁷ cells/ml; or (c) both (a) and (b). In some examples, the use comprises intrathecally administering from 5 to 35 ml of the therapeutic cell suspension. In some examples, the use comprises intrathecally administering from 5 to 30 ml of the therapeutic cell suspension. In some examples, the use comprises intrathecally administering from 5 to 25 ml of the therapeutic cell suspension. In some embodiments, from 10 to 20 ml of the therapeutic cell suspension are administered. In some embodiments, the concentration of cells in the therapeutic cell suspension is from 1×10⁵ to 2×10⁷ cells/ml. In some embodiments, the concentration of cells in the therapeutic cell suspension is from 0.5×10⁶ to 2×10⁷ cells/ml. In some embodiments, the concentration of cells in the therapeutic cell suspension is from 1×10⁶ to 2×10⁷ cells/ml. In some embodiments, the concentration of cells in the therapeutic cell suspension is from 1×10⁶ to 10×10⁶ cells/ml.

All terms, embodiments and definitions disclosed in any one of the above aspects apply and are encompassed herein as well. According to some embodiments, the present invention provides a therapeutic cell suspension for use in a method of treatment of a neurological disease or disorder in a subject in need thereof comprising intrathecally administering from 5 to 25 ml of the therapeutic cell suspension at a rate of from 0.1 to 2 ml/min, wherein the amount of the administered cells is from 1×10⁶ to 5×10⁸. According to some embodiments, the present invention provides a therapeutic cell suspension for use in a method of treatment of a neurological disease or disorder in a subject in need thereof comprising intrathecally administering from 5 to 40 ml of the therapeutic cell suspension at a rate of from 0.1 to 2 ml/min, wherein the concentration of cells in the therapeutic cell suspension is from 1×10⁵ to 2×10⁷ cells/ml. According to some embodiments, the subject is a subject from which a volume of from 5 to 25 ml of cerebrospinal fluid (CSF) had been drawn.

According to some embodiments, the use comprises performing a lumbar puncture. According to some embodiments, from 5 to 35 ml of the therapeutic cell suspension comprising from 1×10⁵ to 2×10⁷ cells/ml are administered. According to some embodiments, from 5 to 25 ml of the therapeutic cell suspension comprising from 1×10⁵ to 2×10⁷ cells/ml are administered. According to some embodiments, from 5 to 25 ml of the therapeutic cell suspension comprising from 1×10⁶ to 2×10⁷ cells/ml are administered. According to some embodiments, the present invention provides a therapeutic cell suspension for use in treating a neurological disease or disorder in a subject in need thereof, wherein the method comprises
(i) drawing a volume from 5 to 40 ml of cerebrospinal fluid (CSF); and
(ii) intrathecally administering from 5 to 40 ml of a therapeutic cell suspension at a rate of from 0.1 to 2 ml/min,

wherein (a) the amount of the administered cells is from 1×10⁶ to 5×10⁸; (b) the concentration of cells in the therapeutic cell suspension is from 1×10⁵ to 2×10⁷ cells/ml; or (c) both (a) and (b). According to some embodiments, the use comprises drawing from 5 to 35 ml of CSF in step (i). According to some embodiments, the use comprises drawing from 5 to 30 ml of CSF in step (i). According to some embodiments, the use comprises drawing from 10 to 20 ml of CSF in step (i). According to some embodiments, the use comprises drawing from 5 to 25 ml of CSF in step (i). According to some embodiments, the use comprises intrathecally administering from 5 to 35 ml of therapeutic cell suspension in step (ii). According to some embodiments, the use comprises intrathecally administering from 5 to 30 ml of C therapeutic cell suspension SF in step (ii). According to some embodiments, the use comprises intrathecally administering from 5 to 25 ml of therapeutic cell suspension in step (ii). According to some embodiments, the use comprises intrathecally administering from 10 to 20 ml of therapeutic cell suspension in step (ii). In some embodiments, the volume of the administered therapeutic cell suspension in step (ii) is equal to or up to ±5 ml of the volume of CSF drawn in step (i). According to some embodiments, the neurological disease or disorder is a neurodegenerative disease or disorder. According to some embodiments, neurodegenerative disease or disorder is selected from the group consisting of a disease of basal ganglia and brain stem, a disease affecting the cerebral cortex, a spinocerebellar degeneration, a degenerative disease affecting motor neurons, or combinations thereof. In some embodiments, the neurodegenerative disease is a disease of a basal ganglia and brain stem. According to some embodiments, the disease is Multiple System Atrophy. According to another embodiment, the disease is Parkinsonism. According to another embodiment, the disease is Idiopathic Parkinson's Disease. According to another embodiment, the disease is Progressive Supranuclear Palsy. According to another embodiment, the disease is Corticobasal Degeneration. According to another embodiment, the disease is Striatonigral Degeneration. According to another embodiment, the disease is Shy-Drager Syndrome. According to another embodiment, the disease is Olivopontocerebellar Atrophy. According to another embodiment, the disease is Huntington's Disease. According to some embodiments, the neurodegenerative disease is Multiple System Atrophy.

According to any one of the above embodiments, the therapeutic cell suspension is formulated for intrathecal administration.

According to some embodiments, the therapeutic cell suspension comprises cells selected from naïve (undifferentiated) adult stem cells, progenitor cells, differentiated cells derived from embryonic or adult stem cells, induced pluripotent stem cells, and combinations thereof. According to some embodiments the therapeutic cell suspension comprises of naïve (undifferentiated) adult stem cells. In some embodiments, the cells are human stem cells. According to other embodiments, the therapeutic cell suspension comprises bone marrow mesenchymal stromal cells. According to some embodiments the therapeutic cell suspension comprises human stem cells derived from the lamina propria of the oral mucosa (hOMSC). According to some embodiments, the cells are derived from the lamina propria of the lining and masticatory oral mucosa. In some embodiments, the cells are derived from the oral mucosa of the gingiva. According to some embodiments, the cells are characterized by simultaneously expressing the following markers: OCT-4, SSEA4, NANOG, SOX2, KLF4, c-MYC, nestin, β-III tubulin, p75, CD29, CD 73, CD90, CD105, and CD166. According to some embodiments, the cells are characterized by simultaneously expressing the following markers KLF4, c-MYC, β-III tubulin, p75 and nestin, and being negative for CD45 and CD31. According to some embodiments, the cells are characterized by simultaneously expressing the following markers: OCT-4, SSEA4, NANOG, SOX2, KLF4, c-MYC, nestin, β-III tubulin, p75, CD29, CD 73, CD90, CD105, and CD166; the cells are negative for CD45 and CD31. According to some embodiments, the cells are isolated cells. According to some embodiments, the cells are pluripotent or multipotent stem cells.

According to some embodiments, the use comprises drawing from 5 to 40 ml of CSF in step (i). According to some embodiments, the use comprises drawing from 5 to 35 ml of CSF in step (i). According to some embodiments, the use comprises drawing from 5 to 30 ml of CSF in step (i). According to some embodiments, the use comprises drawing from 5 to 25 ml of CSF in step (i). According to some embodiments, the use comprises drawing from 5 to 20 ml of CSF in step (i). According to some embodiments, the use comprises drawing from 10 to 20 ml of CSF in step (i). According to some embodiments, the use comprises drawing from 12.5 to 17.5 ml of CSF in step (i). According to some embodiments, the use comprises drawing about 15 ml of CSF in step (i).

According to any one of the above embodiments, the rate of administration of the therapeutic cell suspension is from 0.1 to 1.5 ml/min. According to some embodiments, the rate of administration of the therapeutic cell suspension is from 0.5 to 1.5 ml/min. According to some embodiments, the rate of administration of the therapeutic cell suspension is from 0.6 to 1.3 ml/min. According to some embodiments, the rate of administration of the therapeutic cell suspension is from 0.8 to 1.2 ml/min. According to some embodiments, the rate of administration of the therapeutic cell suspension is about 1 ml/min. According to some embodiments, the administration of therapeutic cell suspension is performed at step (ii). According to some embodiments, the rate of administration of the therapeutic cell suspension at step (ii) is from 0.1 to 1.5 ml/min, from 0.5 to 1.5 ml/min, from 0.6 to 1.3 ml/min, from 0.8 to 1.2 ml/min or about 1 ml/min.

According to some embodiments, the use comprises administering from 1×10⁷ to 1×10⁹ of cells. In some embodiments, the therapeutic cell suspension comprises from 1×10⁶ to 5×10⁸ cells/ml. According to some embodiments, the use (the method) comprises administering from 15×10⁶ to 500×10⁶ of cells. According to some embodiments, the use comprises administering from 20×10⁶ to 150×10⁶ of cells. According to some embodiments, the use comprises administering from 30×10⁶ to 100×10⁶ of cells. According to some embodiments, the use comprises administering from 30×10⁶ to 80×10⁶ of cells. According to some embodiments, the use comprises administering about 37.5×10⁶ cells. According to some embodiments, the use comprises administering about 75×10⁶ cells. According to some embodiments, the cells are hOMSC.

In some embodiments, the therapeutic cell suspension comprises from 0.1×10⁵ to 1×10⁷ cells/ml. In some embodiments, the therapeutic cell suspension comprises from 0.5×10⁵ to 1×10⁷ cells/ml. In some embodiments, the therapeutic cell suspension comprises from 1×10⁵ to 1×10⁷ cells/ml. In some embodiments, the therapeutic cell suspension comprises from 5×10⁵ to 5×10⁶ cells/ml. In some embodiments, the therapeutic cell suspension comprises from 1×10⁶ to 1×10⁷ cells/ml. In some embodiments, the therapeutic cell suspension comprises from 1×10⁶ to 2×10⁷ cells/ml. In some embodiments, the therapeutic cell suspension comprises from 1×10⁶ to 10×10⁶ cells/ml. In some embodiments, the therapeutic cell suspension comprises from 1×10⁶ to 7×10⁶ cells/ml. In some embodiments, the therapeutic cell suspension comprises from 1×10⁶ to 6×10⁶ cells/ml. In some embodiments, the therapeutic cell suspension comprises from 1×10⁶ to 5×10⁶ cells/ml. In some embodiments, the therapeutic cell suspension comprises from 2×10⁶ to 5×10⁶ cells/ml. In some embodiments, the therapeutic cell suspension comprises from 3×10⁶ to 10×10⁶ cells/ml. In some embodiments, the therapeutic cell suspension comprises from 3×10⁶ to 8×10⁶ cells/ml. In some embodiments, the therapeutic cell suspension comprises from 2.5×10⁶ to 4×10⁶ cells/ml. According to some embodiments, the cells are hOMSC.

According to any one of the above embodiments, the volume of the therapeutic cell suspension administered in step (ii) equals to the volume of CSF drawn in step (i). According to any one of the above embodiments, the volume of the therapeutic cell suspension administered in step (ii) is ±1 to 5 ml of the volume of CSF drawn in step (i). According to any one of the above embodiments, the volume of the therapeutic cell suspension administered in step (ii) is ±1 ml, or ±2 ml, or ±3 ml, or ±4 ml, or ±5 ml of the volume of CSF drawn in step (i).

According to any one of the above embodiments, the volume of the therapeutic cell suspension administered in step (ii) is from 5 to 40 ml. According to any one of the above embodiments, the volume of the therapeutic cell suspension administered in step (ii) is from 5 to 35 ml. According to any one of the above embodiments, the volume of the therapeutic cell suspension administered in step (ii) is from 5 to 35 ml. According to any one of the above embodiments, the volume of the therapeutic cell suspension administered in step (ii) is from 5 to 30 ml. According to some embodiments, the volume of the therapeutic cell suspension administered in step (ii) is from 5 to 25 ml. According to any one of the above aspects and embodiments, the volume of the therapeutic cell suspension administered in step (ii) is from 5 to 20 ml. According to any one of the above aspects and embodiments, the volume of the therapeutic cell suspension administered in step (ii) is from 10 to 20 ml. According to any one of the above aspects and embodiments, the volume of the therapeutic cell suspension administered in step (ii) is from 12.5 to 17.5 ml. According to any one of the above aspects and embodiments, the volume of the therapeutic cell suspension administered in step (ii) is about 15 ml.

According to some embodiments, the present invention provides a therapeutic cell suspension comprising hOMSC for use in a method of treatment of a neurodegenerative disease or disorder in a subject in need thereof comprising the steps of:
(i) drawing a volume from 5 to 40 ml of cerebrospinal fluid (CSF); and
(ii) intrathecally administering a therapeutic cell suspension comprising hOMSC at a rate of from 0.1 to 2 ml/min,
   wherein the volume of the administered therapeutic cell suspension is equal to or up to ±5 ml of the volume of CSF drawn in step (i), and wherein (a) the amount of the administered cells is from 10×10⁶ to 200×10⁶, (b) the therapeutic cell suspension comprises from 1×10⁵ to 2×10⁷ cells/ml, or (c) both (a) and (b). In some embodiments, the method comprises drawing from 10 to 20 ml of CSF. In one embodiment, the method comprises drawing from 15 to 30 ml of CSF. In one embodiment, the method comprises drawing from 15 to 25 ml of CSF. In one embodiment, the method comprises drawing about 15 ml of CSF. In one embodiment, the number of administered hOMSC is from 10×10⁶ to 100×10⁶. In one embodiment, the concentration of hOMSC in the therapeutic cell suspension is from 1×10⁶ to 15×10⁶ cells. In one embodiment, the concentration of hOMSC in the therapeutic cell suspension is from 1×10⁶ to 10×10⁶ cells. In one embodiment, the concentration of hOMSC in the therapeutic cell suspension is from 1×10⁶ to 5×10⁶ cells. The combination of several embodiments is contemplated as well. According to some embodiments, the neurodegenerative disease is Multiple System Atrophy.

According to some embodiments, the present invention provides a therapeutic cell suspension for use in a method of treatment of a neurodegenerative disease or disorder in a subject in need thereof the method comprises intrathecally administering 15±5 ml of a therapeutic cell suspension comprising from 20×10⁶ to 100×10⁶ hOMSC at a rate of from 0.1 to 1.2 ml/min. According to some embodiments, the method comprises drawing a volume of 15±5 ml of cerebrospinal fluid (CSF) before administering the therapeutic cell suspension. According to some embodiments, the neurodegenerative disease is Multiple System Atrophy.

According to some embodiments, the present invention provides a therapeutic cell suspension for use in a method of treatment of a neurodegenerative disease or disorder in a subject in need thereof the method comprises the steps of:
(i) drawing a volume of 15±5 ml of cerebrospinal fluid (CSF); and
(ii) intrathecally administering 15±5 ml of a therapeutic cell suspension comprising from 20×10⁶ to 100×10⁶ hOMSC at a rate of from 0.1 to 1.2 ml/min. According to some embodiments, the neurodegenerative disease is Multiple System Atrophy.

According to some embodiments, the present invention provides a therapeutic cell suspension for use in treating of a neurodegenerative disease or disorder in a subject in need thereof comprising the steps of:
(i) drawing a volume of 15±5 ml of cerebrospinal fluid (CSF); and
(ii) intrathecally administering 15±5 ml of a therapeutic cell suspension comprising from 1×10⁶ to 7×10⁶ hOMSC/ml at a rate of from 0.1 to 1.2 ml/min. According to some embodiments, the neurodegenerative disease is Multiple System Atrophy.

According to any one of the above embodiments, the method comprises a lumbar puncture using a spinal needle before drawing CSF or before administering the therapeutic cell suspension.

According to some embodiments, the use is characterized by a lower rate of side effects associated with intrathecal administration of cells in comparison to a corresponding method comprising intrathecal administering cells at a rate higher than 2 ml/min. According to some embodiments, the use is characterized by a lower rate of side effects associated with intrathecal administration of cells in comparison to a corresponding method comprising intrathecal administering of a therapeutic cell suspension comprising more than 2×10⁷ cells/ml at a rate higher than 2 ml/min. According to some embodiments, the side effects are selected from the group comprising of back pain, pain in lower limbs, cell clumping, thickening or mild enhancement of cauda equina nerve roots near the injection site, and any combinations thereof. According to some embodiments, cell clumping comprises cell clumping adjacent to the nerves near the injection site. According to some embodiments, cell clumping comprises cell clumping adjacent to the nerve roots of the cauda equina near the injection site. According to some embodiments, the side effects associated with intrathecal administration of cells are selected from the group comprising of back pain, pain in lower limbs, cell clumping, thickening, or mild enhancement of the cauda equina nerve roots near the injection site, and any combinations thereof. Therefore, according to some embodiments, the method of the present invention causes less of at least one of the following side effects: back pain, pain in lower limbs, cell clumping, thickening or mild enhancement of the cauda equina nerve roots near the injection site. According to one embodiment, the method described in this invention reduces the rate of occurrence of back pain. According to another embodiment, the method described in this invention reduces the rate of occurrence of pain in lower limbs. According to another embodiment, the method described in this invention reduces the rate of occurrence of cell clumping adjacent to the nerve roots of the cauda equina near the injection. According to another embodiment, the method described in this invention reduces the rate of occurrence of thickening of the cauda equina nerve roots near the injection site. According to another embodiment, the method described in this invention reduces the rate of occurrence of enhancement of the cauda equina nerve roots near the injection site.

According to any one of the above aspects and embodiments, the method or the use further comprises a method of preparing a therapeutic cell suspension for injection comprising stem cells prior to the administration of the therapeutic cell suspension.

Therefore, according to some embodiments, the present invention provides a method for treating a neurological disease or disorder in a subject in need thereof comprising preparing a therapeutic cell suspension for injection and intrathecally administering from 5 to 40 ml of the therapeutic cell suspension at a rate of from 0.1 to 2 ml/min, wherein the amount of the administered cells is from 1×10⁶ to 5×10⁸ and/or wherein the concentration of cells in the therapeutic cell suspension is from 0.5×10⁵ to 2×10⁷ cells/ml.

According to other embodiments, the present invention provides a therapeutic cell suspension for use in treating a neurological disease or disorder in a subject in need thereof, wherein the use comprises preparing a therapeutic cell suspension for injection and intrathecally administering from 5 to 40 ml of the therapeutic cell suspension at a rate of from 0.1 to 1.5 ml/min, wherein the amount of the administered cells is from 1×10⁶ to 5×10⁸ and/or wherein the concentration of cells in the therapeutic cell suspension is from 0.5×10⁵ to 2×10⁷ cells/ml.

According to some embodiments, the present invention provides a method of preparing a therapeutic cell suspension for injection comprising stem cells prior to administering it.

According to some embodiments, a method of preparing a therapeutic cell suspension for injection comprising stem cells comprises the steps:
(i) thawing cryopreserved human stem cells e.g. derived from the lamina propria of the oral mucosa (hOMSC) into a solution for injection optionally comprising at least 2 wt % of human serum albumin; and
(ii) washing the cells in the suspension of step (i) with a solution for injection to remove the human serum albumin from the suspension, thereby obtaining the therapeutic cell suspension for injection comprising pluripotent or multipotent stem cells.

According to some embodiments, the present invention provides a therapeutic cell suspension for use in treating a neurological disease or disorder in a subject in need thereof, wherein the use comprises:
(i) thawing cryopreserved human stem cells e.g. derived from the lamina propria of the oral mucosa (hOMSC) into a solution for injection comprising at least 2 wt % of human serum albumin;
(ii) washing the cells in the suspension of step (i) with a solution for injection to remove the serum albumin from the suspension (and cells), thereby obtaining the therapeutic cell suspension; and
(iii) intrathecally administering from 5 to 40 ml of the therapeutic cell suspension obtained in step (ii) at a rate of from 0.1 to 1.5 ml/min, wherein the amount of the administered cells is from 1×10⁶ to 5×10⁸

According to some embodiments, the present invention provides a therapeutic cell suspension for use in treating a neurological disease or disorder in a subject in need thereof, wherein the use comprises:
(i) thawing cryopreserved human stem cells e.g. derived from the lamina propria of the oral mucosa (hOMSC) into a solution for injection comprising at least from 2 to 20 wt % of human serum albumin (HSA);
(ii) diluting the suspension of step (i) with a solution for injection to obtain a therapeutic cell suspension comprising less than 0.3% HSA; and
(iii) intrathecally administering from 5 to 40 ml of the therapeutic cell suspension obtained in step (ii) at a rate of from 0.1 to 1.5 ml/min, wherein the amount of the administered cells is from 1×10⁶ to 5×10⁸.

According to some embodiments, the human stem cells, such as hOMSC, are cryopreserved in a cryopreserving composition. As used herein, the term “cryopreserving composition” refers to a composition that is suitable for the storage of biological material (e.g. cells, tissues, organs and biological molecules) at temperatures below 4° C. In some embodiments, the cryopreserving composition of the invention is in a frozen state, e.g. at a temperature of less than 0° C. less than −5° C., less than −20° C., less than −60° C., less than −70° C., less than −80° C., less than −90° C., less than −170° C.

The therapeutic cell suspension of the present invention comprises the human stem cells as defined in the application, e.g. hOMSC. The terms “therapeutic cell suspension” and “pharmaceutical composition comprising cells” may be used interchangeably. The term “pharmaceutical composition” as used herein refers to a composition comprising at least one active agent as disclosed herein such as hOMSC optionally formulated together with one or more pharmaceutically acceptable carriers. Formulation of the pharmaceutical composition may be adjusted according to applications. In particular, the pharmaceutical composition may be formulated using a method known in the art so as to provide rapid, continuous or delayed release of the active ingredient after administration to mammals.

The term “solution for injection” refers to any isotonic solution suitable for injection into a human body.

In certain embodiments, the solution for injection is selected from PlasmaLyte, Saline solution, PBS, and Ringer's Lactate. According to one embodiment, the solution for injection is PlasmaLyte 148.

In other embodiments, the solution for injection in step (i) (thawing step) comprises at least 3 wt %, at least 4 wt %, at least 5 wt % or at least 6 wt %, or at least 8 wt % of serum albumin. According to some embodiments, the solution for injection in step (i) is referred to as a “thawing solution”. In some embodiments, the solution for injection in step (i) comprises from 1 to 15% w/v serum albumin. In some embodiments, the solution for injection in step (i) comprises from 1 to 10% w/v serum albumin. In some embodiments, the serum albumin is selected from human serum albumin (HSA) and bovine serum albumin (BSA). In some embodiments, the serum albumin is human serum albumin (HSA). Thus, according to some embodiments, the solution for injection in step (i) comprises at least 3 wt %, at least 4 wt %, at least 5 wt % or at least 6 wt %, or at least 8 wt % of HSA. Thus, according to some embodiments, the solution for injection in step (i) comprises from 1 to 10% w/v of HSA. According to some embodiments, the solution for injection in step (i) comprises from 2 to 9% w/v, from 3 to 8% w/v, from 4 to 7% w/v, from 4 to 6% w/v of HSA. According to some embodiments, the solution for injection in step (i) comprises from 3 to 6 wt % of HSA. According to some embodiments, the solution for injection in step (i) comprises from 1 to 15%, from 2 to 12%, from 5 to 15%, from 7 to 12% or about 10% of HSA. According to some embodiments, the ratio between the cryopreserving composition and the solution for injection comprising HSA in step (i) is from 8:1 to 1:3. According to some embodiments, the ratio between the cryopreserving composition and the solution for injection comprising HSA in step (i) is from 6:1 to 1:2. According to some embodiments, the ratio between the cryopreserving composition and the solution for injection comprising HSA in step (i) is from 5:1 to 1:2. According to some embodiments, the ratio between the cryopreserving composition and the solution for injection comprising HSA in step (i) is from 4:1 to 1:2. According to some embodiments, the ratio between the cryopreserving composition and the solution for injection comprising HSA in step (i) is from 3:1 to 1:2. According to some embodiments, the ratio between the cryopreserving composition and the solution for injection comprising HSA in step (i) is from 2:1 to 1:2. According to some embodiments, the ratio between the cryopreserving composition and the solution for injection comprising HSA in step (i) is from 1.5:1 to 1:1.5. According to some embodiments, the ratio between the cryopreserving composition and the solution for injection comprising HSA in step (i) is from 1.3:1 to 1:1.3, or about 1.2:1.

The terms “substantially devoid”, “essentially devoid”, “devoid”, “does not include” and “does not comprise” may be used interchangeably and refer to a composition that does not include, contain or comprise a particular component, e.g. said composition comprises less than 0.1 wt %, less than 0.01 wt %, or less than 0.001 wt % of the component. In some embodiments, the term devoid contemplates a composition comprising traces of the devoid component.

In additional embodiments, the method comprises repeating the washing steps 1, 2, 3 or 4 times.

According to some embodiments, the washing comprises centrifuging the cells, discarding the supernatant, and diluting the cells in a solution for injection.

According to some embodiments, the solution for injection in the step of washing the cells comprises less than 0.5% of HSA (w/v). According to some embodiments, the solution for injection in the step of washing the cells comprises less than 0.4% of HSA (w/v). According to some embodiments, the solution for injection in the step of washing the cells comprises from 0.01 to 0.5% w/v of HSA. According to some embodiments, the solution for injection in the step of washing the cells comprises from 0.03 to 0.5% w/v, from 0.05 to 0.4% w/v, from 0.1 to 0.4% w/v, or from 0.2 to 0.4% w/v of HSA. According to some embodiments, the solution for injection in the step of washing the cells comprises from 0.01 to 0.5% w/v of HSA. According to some embodiments, the solution for injection in the step of washing the cells comprises from 0.03 to 0.5% w/v, from 0.05 to 0.4% w/v, from 0.1 to 0.4% w/v, or from 0.2 to 0.4% w/v of HSA. According to some embodiments, the solution for injection in the step of washing the cells comprises from 0.03 to 0.3% w/v, from 0.05 to 0.3% w/v, from 0.1 to 0.3% w/v, or from 0.2 to 0.3% w/v of HSA. According to some embodiments, the solution for injection in the step of washing the cells is substantially devoid of HSA.

According to some embodiments, the resulting therapeutic cell suspension comprises less than 0.5% of HSA (w/v). According to some embodiments, the resulting therapeutic cell suspension comprises less than 0.4% of HSA (w/v). According to some embodiments, the resulting therapeutic cell suspension comprises less than 0.3% of HSA (w/v). According to some embodiments, the resulting therapeutic cell suspension comprises from 0.01 to 0.5% w/v of HSA. According to some embodiments, the resulting therapeutic cell suspension comprises from 0.03 to 0.5% w/v, from 0.05 to 0.4% w/v, from 0.1 to 0.3% w/v, or from 0.2 to 0.3% w/v of HSA. According to some embodiments, the resulting therapeutic cell suspension comprises from 0.03 to 0.4% w/v, from 0.05 to 0.3% w/v, from 0.01 to 0.3% w/v, from 0.01 to 0.2% w/v of HSA. According to some embodiments, the resulting therapeutic cell suspension is devoid of HSA.

According to some embodiments, the resulting therapeutic cell suspension comprises less than 0.5% of a cryopreserving agent. According to some embodiments, the resulting therapeutic cell suspension comprises less than 0.4% of a cryopreserving agent. The terms “cryopreserving agents” and “cryopreservative agents” may be used herein interchangeably and refer to an agent used to preserve cell viability when cooling to sub-zero centigrade temperatures. According to some embodiments, the cryopreserving agent is DMSO.

According to any one of the above embodiments, the therapeutic cell suspension is sterile.

According to any one of the above embodiments, the preparation of the therapeutic cell suspension for injection is performed in aseptic conditions.

According to one embodiment, the present invention provides a method of preparing a therapeutic cell suspension for injection comprising stem cells, wherein, the method comprises the following steps:
(i) thawing cryopreserved hOMSC into PlasmaLyte 148 comprising at least 4 wt % of HSA; and
(ii) washing the cells in the suspension of step (i) 1, 2 or 3 times with PlasmaLyte 148 substantially devoid of the HSA.

According to some embodiments, the content of HSA in PL in step (i) is from 4 to 7%.

According to one embodiment, the present invention provides a method of preparing a therapeutic cell suspension for injection comprising stem cells, wherein, the method comprises the following steps:
(i) thawing cryopreserved hOMSC into PlasmaLyte 148 comprising at least 4 wt % of HSA; and
(ii) washing the cells in the suspension of step (i) 1, 2 or 3 times with PlasmaLyte 148 substantially comprising devoid of HSA.

According to some embodiments, the content of HSA in PL in step (i) is from 4 to 7%.

According to one embodiment, the present invention provides a therapeutic cell suspension for use in treating a neurological disease or disorder in a subject in need thereof, wherein the use comprises:
(i) thawing cryopreserved hOMSC into PlasmaLyte 148, either comprising from 4 wt % to 10% of HSA;
(ii) washing the cells in the suspension of step (i) 1, 2 or 3 times with PlasmaLyte 148 comprising less than 0.3% w/v HSA, and
(iii) intrathecally administering from 5 to 40 ml of the therapeutic cell suspension obtained in step (ii) at a rate of from 0.1 to 1.5 ml/min, wherein the amount of the administered cells is from 1×10⁶ to 5×10⁸.

According to some embodiments, a method of preparing a therapeutic cell suspension for injection comprising stem cells comprises a steepest dilution of the cryopreserving composition comprising the stem cells with PlasmaLyte. According to some embodiments, the method comprises 2 steps: step (1) comprises thawing the cryopreserving composition comprising the stem cells into PlasmaLyte either comprising from 4 to 10% HSA or not, and step (2) comprises further dilution of the composition obtained in the previous step to obtain a therapeutic cell suspension for injection comprising less than 0.5% HSA.

According to some embodiments, the cryopreserving composition comprises at least 1×10⁶, at least 1.5×10⁶, at least 2×10⁶, at least 2.5×10⁶, at least 5×10⁶, at least 1×10⁷, at least 2×10⁷, or at least 3×10⁷ cells/ml. According to some embodiments, the cryopreserving composition comprises at least 4×10⁷ cells/ml. According to some embodiments, the cryopreserving composition comprises at least 5×10⁷ cells/ml. According to some embodiments, the cryopreserving composition comprises at least 6×10⁷ cells/ml. According to some embodiments, the cryopreserving composition comprises at least 7×10⁷ cells/ml. According to some embodiments, the cryopreserving composition comprises at least 8×10⁷ cells/ml. According to some embodiments, the cryopreserving composition comprises at least 9×10⁷ cells/ml. According to some embodiments, the cryopreserving composition comprises at least 1×10⁸ cells/ml. According to some embodiments, the cryopreserving composition comprises at least 1.2×10⁸ cells/ml. According to some embodiments, the cryopreserving composition comprises at least 1.5×10⁸ cells/ml. According to some embodiments, the cryopreserving composition comprises at least 2×10⁸ cells/ml. According to some embodiments, the cryopreserving composition comprises from 1×10⁶ to 5×10⁸ cells/ml/ml. According to some embodiments, the cryopreserving composition comprises from 1×10⁷ to 1.2×10⁸ cells/ml. According to some embodiments, the cryopreserving composition comprises from 10×10⁶ to 100×10⁶ cells/ml. According to some embodiments, the cryopreserving composition comprises from 30×10⁶ to 100×10⁶ cells/ml. According to some embodiments, the cryopreserving composition comprises from 50×10⁶ to 80×10⁶ cells/ml. According to some embodiments, the cryopreserving composition comprises from 1.5×10⁷ to 1×10⁸ cells/ml. According to some embodiments, the cryopreserving composition comprises from 10×10⁶ to 100×10⁶ cells/ml.

According to some additional embodiments, the resulting therapeutic cell suspension for injection comprises from 1×105 to 1×10⁷ cells/ml. In some embodiments, the therapeutic cell suspension comprises from 5×10⁵ to 50×10⁶ cells/ml. In some embodiments, the therapeutic cell suspension comprises from 1×10⁶ to 1×10⁷ cells/ml. In some embodiments, the therapeutic cell suspension comprises from 1×10⁶ to 7×10⁶ cells/ml.

As discussed above, the method of the present invention using HSA in the process of thawing the cells provides a much higher rate of viable and recovered cells. Using two steps (i.e. thawing into a small amount of a solution for injection, either comprising HSA or not) further increases the viability of cells. According to any one of the above embodiments, at least 85% of the cells in the resulting therapeutic cell suspension are viable cells. According to any one of the above embodiments, at least 90% of the cells in the resulting therapeutic cell suspension are viable cells. According to any one of the above embodiments, at least 92% of the cells in the resulting therapeutic cell suspension are viable cells. According to some embodiments, the cells are viable after 2, 3, or 4 hours when maintained at a temperature of 2-8° C. According to some embodiments, the cells are viable after 5 or 6 hours when maintained at a temperature of 2-8° C.

According to any one of the above embodiments, the method of thawing/preparing the cells provides at least a 10% higher amount of viable cells than a method devoid of step (i). According to any one of the above embodiments, the method provides at least 15% or at least 20% or at least 30% higher amount of viable cells than a method devoid of step (i).

According to some embodiments, at least 60% or at least 65% of cells are recovered from cryopreservation. According to some embodiments, at least 70% of cells are recovered from cryopreservation. According to some embodiments, from 60 to 80% or from 60 to 75% of cells are recovered from cryopreservation. In further embodiments, the cells in the therapeutic cell suspension for injection are stable for at least 6 hours at a temperature of 2-8° C.

According to any one of the above embodiments, the method for preparing the therapeutic cell suspension provides at least a 10% higher rate of recovery from cryopreservation than a method devoid of a 2-step preparation as described hereinabove. According to any one of the above embodiments, the method provides at least 15% or at least 20% or at least 30% higher rate of recovery from cryopreservation than a method devoid of 2-step preparation step as described hereinabove.

According to some embodiments, the present invention provides the method for preparing a therapeutic cell suspension as described per se. According to some embodiments, the present invention further provides a therapeutic cell suspension prepared by a method according to any one of the above embodiments.

According to other embodiments, preparing a therapeutic cell suspension for injection comprising stem cells comprises thawing cryopreserved human stem cells e.g. derived from the lamina propria of the oral mucosa (hOMSC) into a solution for injection. Thus, according to some embodiments, the present invention provides a therapeutic cell suspension for use in treating a neurological disease or disorder in a subject in need thereof, wherein the use comprises thawing cryopreserved human stem cells into from 5 to 40 ml of a solution for injection thereby obtaining a therapeutic cell suspension for injection and intrathecally administering said therapeutic cell suspension at a rate of from 0.1 to 1.5 ml/min, wherein the amount of the administered cells is from 1×10⁶ to 5×10⁸. According to some embodiments, the composition comprising cryopreserved human stem cells comprises up to 4 wt % DMSO. According to some embodiments, the composition comprising cryopreserved human stem cells comprises up to 3 wt % DMSO. According to some embodiments, the composition comprising cryopreserved human stem cells comprises up to 2 wt % DMSO. According to some embodiments, the composition comprising cryopreserved human stem cells comprises up to 4 wt % DMSO. According to some embodiments, the composition comprising cryopreserved human stem cells comprises from 1 to 4 wt %, from 1 to 3 wt %, or from 1.5 to 2.5 wt % DMSO. According to some embodiments, the resulting therapeutic cell suspension comprises less than 0.4 wt % DMSO. According to some embodiments, the resulting therapeutic cell suspension comprises less than 0.35 wt % DMSO. According to some embodiments, the resulting therapeutic cell suspension comprises less than 0.3 wt % DMSO. According to some embodiments, the resulting therapeutic cell suspension comprises less than 0.25 wt % DMSO. According to some embodiments, the resulting therapeutic cell suspension comprises less than 0.2 wt % DMSO. According to some embodiments, the resulting therapeutic cell suspension comprises less than 0.15 wt % DMSO. According to some embodiments, the resulting therapeutic cell suspension comprises less than 0.1 wt % DMSO. It would be clear to any skilled person to calculate the required dilution based on the initial concentration of DMSO and the desired one.
Clause 1. A method for treating a neurological disease or disorder in a subject in need thereof comprising the steps of:
   (i) performing a lumbar puncture; and
   (ii) intrathecally administering from 5 to 40 ml of a therapeutic cell suspension at a rate of from 0.1 to 1.5 ml/min,
   wherein the amount of the administered cells is from 1×10⁶ to 5×10⁸.
Clause 2. The method of any clauses herein, particularly clause 1, wherein the method further comprises drawing a volume from 5 to 40 ml of cerebrospinal fluid (CSF) at step (i) and wherein the volume of the administered therapeutic cell suspension is equal to or up to ±5 ml of the volume of CSF drawn in step (i).
Clause 3. The method of any clauses herein, particularly clause 1 or 2, wherein the neurological disease or disorder is a neurodegenerative disease.
Clause 4. The method of any clauses herein, particularly clauses 1-3, wherein the neurodegenerative disease is selected from the group comprising of Multiple System Atrophy, Alzheimer's Disease, Pick Disease, Parkinsonism, Idiopathic Parkinson's Disease, Progressive Supranuclear Palsy, Corticobasal Degeneration, Striatonigral Degeneration, Shy-Drager Syndrome, Olivopontocerebellar Atrophy, Huntington Disease, Spinocerebellar Ataxias, Friedreich Ataxia, Ataxia-Telangiectasia, Amyotrophic Lateral Sclerosis, Bulbospinal Atrophy, Spinal Muscular Atrophy, and combinations thereof.
Clause 5. The method of any clauses herein, particularly clause 3, wherein the neurological disease or disorder is selected from the group comprising of trauma to the brain and or to the spinal cord and or the spinal ganglions, chronic diseases that affect the central nervous system as for example diabetes, lupus, post-atherosclerotic stroke and post-hemorrhagic stroke, is caused by intervertebral disc pathologies.
Clause 6. The method of any clauses herein, particularly clauses 1-4, characterized by at least one of: (i) wherein the rate of administration of the therapeutic cell suspension at step (ii) is from 0.5 to 1.5 ml/min; (ii) the method comprises intrathecally administering from 5 to ml of a therapeutic cell suspension; and (iii) the method further comprises drawing a volume from 5 to ml of cerebrospinal fluid (CSF) at step (i) and wherein the volume of the administered therapeutic cell suspension is equal to or up to ±5 ml of the volume of CSF drawn in step (i).
Clause 7. The method of any clauses herein, particularly clause 1, wherein the method comprises administering from 1×10⁷ to 1×10⁸ cells.
Clause 8. The method of any clauses herein, particularly clauses 1-7, wherein the volume of the therapeutic cell suspension administered in step (ii) essentially equals to the volume of CSF drawn in step (i).
Clause 9. The method of any clauses herein, particularly clause 1, wherein the concentration of cells in the therapeutic cell suspension is from 0.5×10⁵ to 2×10⁷ cells/ml.
Clause 10. The method of any clauses herein, particularly clause 1, wherein the method is characterized by a lower rate of side effects associated with intrathecal administration of cells in comparison to a corresponding method comprising intrathecal administering cells at a rate higher than 2 ml/min or in comparison to a corresponding method comprising intrathecal administering a therapeutic cell suspension comprising more than 2×10⁷ cells/ml at a rate higher than 2 ml/min.
Clause 11. The method of any clauses herein, particularly clause 10, wherein the side effects are selected from the group comprising of back pain, pain in lower limbs, clumping of the administered cells adjacent to the nerve roots, thickening, or mild enhancement of cauda equina nerve roots near the injection site, and any combinations thereof.
Clause 12. The method of any clauses herein, particularly clause 1, wherein the therapeutic cell suspension comprises a plurality of cells selected from the group consisting of human stem cells, naïve (undifferentiated) adult stem cells, progenitor cells, differentiated cells derived from embryonic or adult stem cells, induced pluripotent stem cells and their derivatives, and combinations thereof.
Clause 13. The method of any clauses herein, particularly clause 12, wherein cells are the human stem cells.
Clause 14. The method of any clauses herein, particularly clause 13, wherein the human stem cells are derived from lamina propria of the oral mucosa.
Clause 15. The method of any clauses herein, particularly clause 14, wherein the cells are derived from the lamina propria of the lining and masticatory oral mucosa.
Clause 16. The method of any clauses herein, particularly clause 15, wherein said cells are characterized by simultaneously expressing the following markers: Oct-4, SSEA4, Nanog, Sox2, KLF4, c-MYC, nestin, β-III tubulin, p75, CD29, CD 73, CD90, CD105, and CD166.
Clause 17. The method of any clauses herein, particularly clause 14, wherein said cells are characterized by simultaneously expressing the following markers: KLF4, c-MYC nestin, p75, and β-III tubulin, and wherein the cells are negative for CD45 and CD31.
Clause 18. The method of any clauses herein, particularly clause 1, wherein (i) the therapeutic cell suspension is administered via the spinal needle used for the lumbar puncture; (ii) the therapeutic cell suspension is mixed during the administration to prevent clumping of cells, or (iii) both (i) and (ii).
Clause 19. The method of any clauses herein, particularly clause 18, wherein the mixing comprises rotating or tilting the device comprising the therapeutic cell suspension.
Clause 20. A method for intrathecally administering a therapeutic cell suspension to a subject in need thereof comprising the steps of:
   (i) performing a lumbar puncture using a spinal needle and drawing a volume of from 5 to 40 ml of cerebrospinal fluid (CSF); and
   (ii) intrathecally administering a therapeutic cell suspension at a rate of from 0.1 to 1.5 ml/min, wherein the volume of the administered therapeutic cell suspension is equal to or about ±5 ml of the volume of CSF drawn in step (i), and wherein the amount of the administered cells is from 1×10⁶ to 5×10⁸.
Clause 21. A therapeutic cell suspension for use in treating a neurological disease or disorder in a subject in need thereof, wherein the use comprising the steps of:
   (i) performing a lumbar puncture; and
   (ii) intrathecally administering from 5 to 40 ml of a therapeutic cell suspension at a rate of from 0.1 to 1.5 ml/min,
wherein the amount of the administered cells is from 1×10⁶ to 5×10⁸.
Clause 22. The method or the therapeutic cell suspension for use of any clauses herein, particularly clauses 1 to 21, further comprises preparing the therapeutic cell suspension prior to its administration, wherein preparing the therapeutic cell suspension comprises the steps:
(i) thawing cryopreserved human stem cells into a solution for injection comprising at least 2 wt % of human serum albumin (HSA); and
(ii) washing the cells in the suspension of step (i) with a solution for injection devoid of the serum albumin to remove the serum albumin from the cells/suspension, thereby obtaining the therapeutic cell suspension human stem cells.
Clause 23. The method or the therapeutic cell suspension for use of any clauses herein, particularly clauses 1 to 21, further comprises preparing the therapeutic cell suspension prior to its administration, wherein preparing the therapeutic cell suspension comprises thawing human stem cells cryopreserved with from 1 to 4 wt % DMSO into a solution for injection thereby obtaining the therapeutic cell suspension, wherein the resulting therapeutic cell suspension comprises less than 0.4 wt % DMSO.
Clause 24. The method or the therapeutic cell suspension for use of any clauses herein, particularly clauses 22 and 23, wherein the solution for injection is selected from PlasmaLyte 148, Saline solution, and Ringer's Lactate.
Clause 25. The method or the therapeutic cell suspension for use of any clauses herein, particularly clauses 22-24, wherein the solution for injection comprises in step (i) at least 3 wt %, at least 4 wt %, at least 5 wt % or from 3 to 6 wt % of human serum albumin.
Clause 26. The method or the therapeutic cell suspension for use of any clauses herein, particularly clauses 22-25, wherein the washing step is repeated 2, 3 or 4 times.
Clause 27. The method or the therapeutic cell suspension for use of any clauses herein, particularly clauses 22-26, wherein the thawing of step (i) is performed at 37° C.
Clause 28. The method or the therapeutic cell suspension for use of any clauses herein, particularly clauses 22-27, comprising:
   (i) thawing cryopreserved hOMSC in PlasmaLyte 148 comprising at least 4 wt % of HSA; and
   (ii) washing the cells in the suspension of step (i) 1, 2 or 3 times with PlasmaLyte 148 devoid of the serum albumin.
Clause 29. The method or the therapeutic cell suspension for use of any clauses herein, particularly clauses 22-28, wherein the resulting therapeutic cell suspension for injection comprises at least 90% viable cells.
Clause 30. The method or the therapeutic cell suspension for use of any clauses herein, particularly clauses 22-29, wherein at least 60% or at least 65% of cells are recovered from the cryopreservation.
Clause 31. The method or the therapeutic cell suspension for use of any clauses herein, particularly clauses 22-30, wherein the cells in the therapeutic cell suspension for injection are stable for at least 6 hours at the temperature of 2-8° C.
Clause 32. The method or the therapeutic cell suspension for use of any clauses herein, particularly clauses 22-31, wherein the resulting therapeutic cell suspension for injection is substantially devoid of the HSA.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### EXAMPLES

### Example 1. Thawing Stem Cells in the Presence of Human Serum Albumin

### Materials and Methods

The used reagents are described in Table 1.

TABLE 1
Reagents and Media

| Material | Volume | |
|---|---|---|
| Refrigerated PlasmaLyte 148 (PL) | 1 | L |
| 20% HSA | 1 | bottle |
| Sterile water or PBS for thawing | 500 | ml |

| | |
|---|---|
| hOMSC | As needed |

Each 1000 mL of Plasmalyte 148, pH 7.4 infusion solution contains Sodium chloride 5.26 g Sodium gluconate 5.02 g Sodium acetate trihydrate 3.68 g Potassium chloride 370 mg Magnesium chloride hexahydrate 300 mg.

Preparation of hOMSC Drug Product (DP) Formulation

The cryovials (0.5-1.5 ml) were placed in the 37° C. pre-warmed water box and continuously stirred while thawing. The cell suspension from the vial(s) was transferred into the 50 ml tube(s) containing the PL+3-10% human serum albumin (HSA) or PL, which were then centrifuged at 4° C. Then, the supernatant was aspirated. The cells were carefully suspended with a total of 10-20 ml of refrigerated PL in one 50 ml tube.

### Cell Counting

Cells were counted as known in the art. Cell viability was ≥80%.

### Experimental Procedure

The tested objective was the contribution of initial thawing solutions, PlasmaLyte (PL) solution or PL containing 5% Human Serum Albumin (HSA), to the viability and stability of the cells in the final formulation.

The cells were thawed in the tested solutions (PL with or without HSA), washed once in PL and formulated into the final product.

The two thawing protocols were both initiated with ˜10⁷ cells/mL cryopreserved in Cryostor 5 solution. The first cell sampling was made immediately after thawing cells in either PL or in PL+3-10% HSA, at 37° C., after which the vials were centrifuged. The second cell sampling was performed after draining the supernatant and washing in 20 ml PL at 4° C., for both protocols. The third cell sampling was performed after the cells were diluted to the desired concentration (1×10⁶-5×10⁶ cells/ml, for both protocols). The fourth and fifth cell samplings were performed at 4° C., after 1 and 3 hours, respectively. These two steps were identical for both protocols. The results are elaborated in Table 2.

TABLE 2
Thawing protocol test results

| | 1st sampling (in | | | | |
|---|---|---|---|---|---|
| | tested thawing | | 3rd sampling | 4th sampling | 5th sampling |
| | solution) | | T0 (cell/ml) | 1 hrs (cell/ml) | 3 hrs (cell/ml) |
| % cell viability | 92 | 89 | 87 | 87 | 85 |
| % recovery | 59 | 57 | | | |
| from cryo | | | | | |
| % cell viability | 95 | 95 | 95 | 93 | 92 |
| % recovery | 92 | 72 | | | |
| from cryo | | | | | |

As evident from the results, PL+HSA solution prevented a large loss of cells during their thawing (92% vs 59%) and sustains stable cell viability during the thawing procedure until final product formulation (95% vs 87%).

### Example 2

Intrathecal therapeutic cell suspensions denoted hOMSC composition were prepared as follows.

Cryovials comprising about 2 ml of human oral mucosal stem cells (hOMSC), at a concentration of 107-10⁸ cells/mL frozen in a cryoprotecting composition were each thawed and dissolved in PlasmoLyle 148 (PL). The final volume of the therapeutic cell suspension was 15 ml and comprised 30-120×10⁶ cells.

In other examples, the final volume of the therapeutic cell suspension is 5-20 ml and comprises 10-200×10⁶ cells.

### Storage

The DP bag is kept in a temperature-controlled environment (2-8° C.) until release test results are available. A total of 75±15×10⁶ or 37.5±7.5×10⁶ cells were administered in a single Intrathecal (IT) injection, following the clinical study protocol and the following instructions:

Administration of hOMSC:

The procedure is performed by a trained and experienced physician in reaching the intrathecal space by lumbar puncture (LP). The procedure is performed as follows:
1. Using a sterile technique, a spinal needle is inserted in the subject's lumbar region through the skin and between two of the lower lumbar vertebrae (e.g., L3-L4, L4-L5; L5-S1). A 22G or 23G Sprotte Needle or equivalent is used for penetration of dura and collection of CSF.
2. After reaching the intrathecal space, the puncture needle is left in place until 10-20 ml of CSF are collected.
3. A 20 ml syringe containing 10-20 ml of hOMSC cell suspension (Product Syringe) is connected to the puncture needle by a catheter to facilitate administration. The cell suspension is injected at a rate of 0.5-1.5 ml/minute over 10-15 minutes. During the product administration, the syringe is inverted constantly to maintain cell suspension homogeneity and to ensure that there are no visible clumps prior to administration.
4. The puncture needle is left in place for additional 5 minutes and then removed.

### Example 3. Formulation of Thawed Cells into Final Product

Cells, (human oral mucosa stem cells (hOMSC) were harvested and cryopreserved (cryo) with 5% DMSO at 25×10⁶ viable cells/vial. Two parallel thawing protocols were tested.

Cells (3 or 4 vials) were each thawed into 20 ml of either PlasmaLyte 148 (PL) or in PL+5% human serum albumin (HSA), at 37° C., after which the vials were centrifuged. The cells were centrifuged and after draining the supernatant was washed with about 15 ml PL devoid of HSA to reach the concentration of ˜5×10⁶ cells/ml. Overall, the cell suspension comprises about 80×10⁶ cells.

Administration of hOMSC:

The procedure was performed by a trained and experienced physician in reaching the intrathecal space by lumbar puncture (LP). The procedure was performed in four subjects suffering from multiple system atrophy as follows:
1. A lumbar puncture (LP) was performed by inserting a spinal needle in the subject's lumbar region through the skin and between two of the lower lumbar vertebrae (e.g., L3-L4, L4-L5; L5-S1). A 22G or 23G Sprotte Needle or equivalent is used for penetration of dura and collection of CSF.
2. After reaching the intrathecal space, the puncture needle is left in place until about 15 ml of CSF were collected.
3. A 20 ml syringe containing 15 ml of hOMSC cell suspension (Product Syringe) was connected to the puncture needle by a catheter to facilitate administration. The cell suspension was injected at a rate of about 1 ml/minute over 15 minutes. During the product administration, the syringe was inverted constantly to maintain cell suspension homogeneity and to ensure that there are no visible clumps prior to administration. The puncture needle is left in place for additional 5 minutes and then removed.

The patients did not report any side effects that are usually associated with IT administration of cell suspensions such as back pain, pain in lower limbs, cell clumping adjacent to the nerve root, thickening or mild enhancement of cauda equina nerve roots as observed e.g. by the MRI scans.

In another example, CSF is drawn from Cisterna magna and the suspension of hOMSC is administered via lumbar puncture.

### Example 4. Formulation of Thawed Cells into Final Product

Cells, (human oral mucosa stem cells (hOMSC) were harvested and cryopreserved (cryo) with 2% DMSO at ˜70×10⁶ viable cells/vial. Two parallel thawing protocols were tested.

Cells were each thawed into 15-20 ml of either PlasmaLyte 148 (PL) or in PL+3-10% human serum albumin (HSA), at 37° C., after which the vials were centrifuged or not. For the sample in which the cells were centrifuged, after draining the liquid fraction, the supernatant was washed with about 15 ml PL devoid of HSA to reach the desired concentration (1×10⁶-5×10⁶ cells/ml, for both protocols).

### Example 5. Clinical Trials

In a non-controlled clinical study, 25×10⁶-75×10⁶ hOMSC cells in 15 ml were administered intrathecally at a rate of from 0.1 to 1.5 ml/min. No serious adverse effects related both to the product or to the mode of administration were reported.

### Example 6. Intrathecal Administration of Stem Cells

Stem cells, such as bone marrow mesenchymal stromal cells, neural crest derived stem cells, or embryonic neural crest derived stem cells, are harvested and cryopreserved (cryo) with 5% DMSO at 75×10⁶ viable cells/vial.

The cells are thawed as described in Example 3 or 4 and intrathecally administered as described in examples 2, 3 and 5.

Human bone marrow mesenchymal stromal cells are thawed and 25 ml of a composition comprising 20-150×10⁶ cells and less than 0.5 wt % DMSO is prepared. About 25 ml of CSF are drawn through a needed inserted between verterea at the region L3-L5. The composition (about 25 ml) comprising stem cells is then intrathecally administered at a rate of 0.5-1.5 ml/minute over 10-15 minutes.

Neural crest derived stem cells are thawed and 25 ml of a composition comprising 20-150×10⁶ cells and less than 0.5 wt % DMSO is prepared. About 25 ml of CSF are drawn through a needed inserted between verterea at the region L3-L5. The composition (about 25 ml) comprising stem cells is then intrathecally administered at a rate of 0.5-1.5 ml/minute over 10-15 minutes.

The cells are administered for treatment of neurodegenerative diseases.

Although the present invention has been described herein above by way of preferred embodiments thereof, it can be modified, without departing from the spirit and nature of the subject invention as defined in the appended claims.

1. A method for treating a neurological disease or disorder in a subject in need thereof, the method comprising:
intrathecally administering from 5 ml to 40 ml of a therapeutic cell suspension at a rate of from 0.1 ml/min to 1.5 ml/min, wherein the amount of the administered cells is from 1×10⁶ to 5×10⁸.

2. The method of claim 1, further comprising a step of performing a lumbar puncture prior to intrathecally administering the therapeutic cell suspension.

3. The method of claim 1, further comprising drawing a volume of from 5 to 40 ml of cerebrospinal fluid (CSF) prior to intrathecally administering the therapeutic cell suspension, wherein the volume of the administered therapeutic cell suspension is equal to or up to ±5 ml of the volume of drawn CSF.

4. The method of claim 1, wherein the neurological disease or disorder is a neurodegenerative disease.

5. The method to claim 4, wherein the neurodegenerative disease is selected from the group comprising of Multiple System Atrophy, Alzheimer's Disease, Pick Disease, Parkinsonism, Idiopathic Parkinson's Disease, Progressive Supranuclear Palsy, Corticobasal Degeneration, Striatonigral Degeneration, Shy-Drager Syndrome, Olivopontocerebellar Atrophy, Huntington Disease, Spinocerebellar Ataxias, Friedreich Ataxia, Ataxia-Telangiectasia, Amyotrophic Lateral Sclerosis, Bulbospinal Atrophy, Spinal Muscular Atrophy, and combinations thereof.

6. The method of claim 1, wherein the neurological disease or disorder is selected from the group comprising of trauma to the brain and/or to the spinal cord and/or the spinal ganglions, chronic diseases that affect the central nervous system, diabetes, lupus, post-atherosclerotic stroke and post-hemorrhagic stroke caused by intervertebral disc pathologies.

7. The method of claim 1, wherein the method is characterized by at least one of:
(i) the rate of administration of the therapeutic cell suspension is from 0.5 to 1.5 ml/min;
(ii) the method comprises intrathecally administering from 5 to 30 ml of a therapeutic cell suspension; or
(iii) the method further comprises drawing a volume from 5 to 30 ml of cerebrospinal fluid (CSF) prior to intrathecally administering the therapeutic cell suspension, wherein the volume of the administered therapeutic cell suspension is equal to or up to ±5 ml of the volume of drawn CSF.

8. The method of claim 1, comprising administering from 1×10⁷ to 1×10⁸ cells.

9. The method of claim 2, wherein the volume of the administered therapeutic cell suspension essentially equals to the volume of drawn CSF.

10. T The method of claim 1, wherein the concentration of cells in the therapeutic cell suspension is from 0.5×10⁵ to 2×10⁷ cells/ml.

11. The method of claim 1, wherein the method is characterized by a lower rate of side effects associated with intrathecal administration of cells in comparison to a corresponding method comprising intrathecal administering cells at a rate higher than 2 ml/min or in comparison to a corresponding method comprising intrathecal administering a therapeutic cell suspension comprising more than 2×10⁷ cells/ml at a rate higher than 2 ml/min.

12. The method of claim 11, wherein the side effects are selected from the group comprising of back pain, pain in lower limbs, clumping of the administered cells adjacent to the nerve roots, thickening, or mild enhancement of cauda equina nerve roots near the injection site, and any combinations thereof.

13. The method of claim 1, wherein the therapeutic cell suspension comprises a plurality of cells selected from the group consisting of human stem cells, naïve (undifferentiated) adult stem cells, progenitor cells, differentiated cells derived from embryonic or adult stem cells or the progenitors of embryonic stem cells or adult stem cells, induced pluripotent stem cells and their progenies and derivatives, genetically manipulated cells transduced to express specific trophic factors or therapeutic factors, tissue-specific differentiated cells obtained by genetic engineering and combinations thereof.

14. The method of claim 13, wherein cells are the human stem cells.

15. The method of claim 14, characterized by one of more of the following:
(i) the human stem cells are derived from lamina propria of the oral mucosa;
(ii) cells are derived from the lamina propria of the lining and masticatory oral mucosa;
(iii) cells are characterized by simultaneously expressing the following markers: Oct-4, SSEA4, Nanog, Sox2, KLF4, c-MYC, nestin, β-III tubulin, p75, CD29, CD 73, CD90, CD105, and CD166; or
(iv) the cells are characterized by simultaneously expressing the following markers: KLF4, c-MYC nestin, p75, and β-III tubulin, wherein the cells are negative for CD45 and CD31.

16. The method of claim 1, wherein (i) the therapeutic cell suspension is administered via the spinal needle used for the lumbar puncture; (ii) the therapeutic cell suspension is mixed during the administration to prevent clumping of cells or (iii) both (i) and (ii).

17. The method of claim 16, wherein the mixing comprises rotating or tilting the device comprising the therapeutic cell suspension.

18. The method of claim 1, wherein the method further comprises preparing the therapeutic cell suspension prior to its administration, wherein preparing the therapeutic cell suspension comprises:
(i) thawing cryopreserved human stem cells into a solution for injection comprising at least 2 wt % of human serum albumin (HSA); and
(ii) washing the cells in the suspension of step (i) with a solution for injection devoid of the HSA to remove the HSA from the suspension, thereby obtaining the therapeutic cell suspension of human stem cells.

19. The method of claim 1, wherein the method further comprises preparing the therapeutic cell suspension prior to its administration, wherein preparing the therapeutic cell suspension comprises thawing human stem cells cryopreserved with from 1 to 4 wt % DMSO into a solution for injection thereby obtaining the therapeutic cell suspension, wherein the resulting therapeutic cell suspension comprises less than 0.4 wt % DMSO.

20. The method of claim 1, wherein the method further comprises preparing the therapeutic cell suspension prior to its administration, wherein preparing the therapeutic cell suspension comprising (i) thawing human stem cells cryopreserved with from 1 to 4 wt % DMSO into a solution for injection comprising human serum albumin (HSA), and (ii) consequent dilution of the suspension obtained in step (i) with into a solution for injection thereby obtaining the therapeutic cell suspension, wherein the resulting therapeutic cell suspension comprises less than 0.4 v/v % DMSO and less than 0.3 v/v HSA.
